(19) United States
(12) Patent Application Publication
(10) Pub. No.: US 2019/0358316 A1
(43) Pub. Date: Nov. 28, 2019

(54) INFECTIOUS BRONCHITIS VIRUS VACCINE USING NEWCASTLE DISEASE VIRAL VECTOR

(71) Applicant: University of Maryland, College Park, College Park, MD (US)

(72) Inventors: Siba K. SAMAL, Hyattsville, MD (US); Edris SHIRVANI, Hyattsville, MD (US)

(21) Appl. No.: 16/483,969

(22) PCT Filed: Feb. 6, 2018
(86) PCT No.: PCT/US18/17029

§ 371 Date: Aug. 6, 2019

Related U.S. Application Data
(60) Provisional application No. 62/455,290, filed on Feb. 6, 2017.

(51) Int. Cl. A61K 39/17; A61K 39/295; C12N 15/86; A61P 31/14

(52) U.S. Cl. CPC A61K 39/17; A61K 39/295; A61K 2039/5258; A61P 31/14; C12N 15/86

(57) ABSTRACT

Provided are compositions and methods that involve recombinant Newcastle disease viruses (rNDVs) that contain an S protein of infectious bronchitis virus (IBV). The rNDV particles include a contiguous segment of IBV S protein that spans an IBV cleavage site between IBV SI and IBV S2 proteins, and can include a full length IBV S protein. Because the particles are multivalent they also stimulate a protective immune response against NDV infection. The compositions are particularly useful for use with avian animals, such as chickens. Isolated rNDV particles, and vaccine compositions that contain them are also provided.

### CROSS REFERENCE TO RELATED APPLICATIONS

This application claims priority to U.S. Provisional Application No. 62/455,290, filed on Feb. 6, 2017, the disclosure of which is hereby incorporated by reference.

### FIELD OF THE DISCLOSURE

The disclosure generally relates to vaccines. More particularly the disclosure generally relates to modified paramyxoviruses such as recombinant Newcastle disease viruses (rNDVs) expressing proteins of infectious bronchitis virus (IBV) using reverse genetics to form multivalent vaccines.

### BACKGROUND OF THE DISCLOSURE

Infectious Bronchitis (IB) is an acute and highly contagious viral respiratory disease of chickens^{1,2}. IB causes major economic losses in commercial chickens throughout the world^{1,3}. It is one of the most prevalent diseases in the poultry industry. The disease is usually characterized by respiratory signs including gasping, coughing and sneezing. However, the virus can also infect urogenital and reproductive tracts causing renal dysfunction and decreased egg production^{3,4}.

IB is caused by infectious bronchitis virus (IBV), a member of the family Coronaviridae. The genome of IBV is a single stranded, positive-sense RNA of approximately 27.6 kb in length. It encodes a large polyprotein containing spike (S), small envelope (E), membrane (M), nucleocapsid (N) and 15 non-structural proteins. The S glycoprotein is the major antigen against which neutralizing and protective antibodies are produced. The S protein is cleaved into S1 and S2 subunits post translationally⁴. The S1 subunit is responsible for viral attachment to host cell and contains major neutralizing epitopes. The S2 subunit is highly conserved among IBV strains and contributes to viral fusion activity and elicits some minor but broadly reactive neutralizing antibodies⁵⁻⁹.

Currently IB in commercial chickens is controlled by the use of live attenuated and inactivated IBV vaccines. However, current live attenuated vaccine strains are not genetically stable and frequently undergo reversion to virulence^{10,11}. Furthermore, circulation of live-attenuated viruses in the environment provides a setting in which the viral population may undergo mutations and recombination leading to creation of variant viruses^{12,13}. Sequence analysis of pathogenic IBV strains circulating in the field has found live vaccines as one of the sources of outbreak¹⁴. Moreover, live IBV vaccines may causes pathological lesions or secondary bacterial infections in 1-day-old vaccinated chicks^{15,16}. Inactivated IBV vaccines, which usually are administered by injection to layers and breeders at 13 to 18 weeks of age are not an alternative for live-attenuated IBV vaccines. Inactivated vaccines of IBV do not elicit strong immune responses in chickens against circulating virulent IBV strains^{17,18}. The production and administration of inactivated vaccines are also time consuming and costly^{2,10.} Therefore, there is a great need to develop an alternative IBV vaccine. The present disclosure is pertinent to this need.

### SUMMARY OF THE DISCLOSURE

The present disclosure provides a demonstration of using reverse genetics to generate recombinant Newcastle disease viruses (rNDVs) expressing S1, S2 and S proteins of IBV strain Mass-41. The protective efficacies of rNDVs expressing S1, S2 or S protein of IBV were compared in chickens against a virulent IBV strain Mass-41 challenge. The results demonstrate that including the whole S protein is superior to including either S1 or S2 separately as a protective antigen of IBV. The data also demonstrate that rNDV strain LaSota expressing the S protein of IBV strain Mass-41 protects chickens against both virulent IBV and virulent NDV challenges, thereby demonstrating a bivalent vaccine approach. Thus, in embodiments the disclosure provides a method comprising administering an immunologically effective amount of rNDV particles to avian animals to stimulate a protective immune response against IBV, wherein the rNDV particles comprises a contiguous segment of IBV S protein that spans an IBV cleavage site between IBV S1 and IBV S2 proteins. In an embodiment the contiguous segment of IBV S protein comprises a full length IBV S protein. In certain implementations, a protective immune response stimulated by compositions and methods of this disclosure is greater than a reference value obtained from administration of rNDV particles that comprise only an intact S1 or an intact S2 IBV protein. The protective immune response can be determined by a variety of measures, such as by using a severity score for respiratory clinical signs of IBV infection. In certain embodiments, the protective immune response is serologically distinguishable from an immune response to infection by unmodified IBV. In certain approaches, RNA encoding the IBV S protein is codon optimized for expression of the IBV S protein in chicken cells. Such optimization for translated RNA can be configured by engineering the viral genome to result in codon-optimized mRNA that can be optimized for expression in, for example, chicken cells.

Due to the multivalent characteristic of the rNDV particles, the rNDV particles also stimulate a protective immune response against NDV infection. The diversity of the immune response can be expanded by providing IBV S protein as a chimeric protein that further comprises at least a second polypeptide sequence from a pathogen that is not NDV or IBV. This configuration forms at least a multivalent immunogenic agent, wherein the second polypeptide stimulates a protective immune response to the pathogen that is not NDV or IBV. Accordingly, bivalent, trivalent, and higher numbers of distinct immunogenic determinants can be comprised by the rNDV particles of this disclosure.

In certain approaches the rNDV particles are administered to an avian animal that is an embryo, a fledgling, or an adult avian animal. In embodiments, the avian animal is a chicken, such as Gallus gallus. In embodiments, populations or sub-populations of avian animals are vaccinated to promote, for example, herd immunity.

The disclosure includes a plurality of isolated rNDV particles, and compositions comprising them, such as in a vaccine formulation, which may comprise an adjuvant. The particles may also be present in, for example, an avian embryo. In embodiments, the rNDV particles can be characterized by having a segment of the RNA genome that enables production of the S protein is not mutated over at least five avian embryo passages.

### BRIEF DESCRIPTION OF THE FIGURES

For a fuller understanding of the nature and objects of the disclosure, reference should be made to the following detailed description taken in conjunction with the accompanying figures.

FIG. 1 shows a schematic diagram of recombinant NDV constructs containing IBV genes. Seven transcription cassettes including; 1) Four versions of codon optimized S1 subunit of S gene of IBV strain Mass-41, namely, a) codon optimized S1 subunit of S gene of IBV strain Mass-41 (1611 nt), b) codon optimized S1 subunit of S gene of IBV strain Mass-41 (1611 nt) fused with N-terminus of transmembrane and cytoplasmic tail of F gene of IBV strain Mass-41 (255 nt), c) codon optimized S1 subunit of S gene of IBV strain Mass-41 (1611 nt) containing putative cleavage site residues of S1 gene of IBV strain Mass-41 fused with N-terminus of transmembrane and cytoplasmic tail of F gene of NDV (171 nt) and d) codon optimized S1 gene of IBV strain Mass-41 (1593 nt) without cleavage site residues of S1 gene of IBV strain Mass-41 fused with N-terminus of transmembrane and cytoplasmic tail of F gene of NDV (171 nt), 2) The N-terminus of codon optimized S2 gene of IBV strain Mass-41 (1878 nt) fused with C-terminus of signal peptide sequence of S gene of IBV strain Mass-41 (69 nt), 3) The codon optimized S gene of IBV strain Mass-41 (3489 nt) and 4) The non-codon optimized S gene of IBV strain Mass-41 (3489 nt) were flanked by genetic elements required for a transcriptional cassette and cloned into antigenomic cDNA of LaSota between P and M genes using PmeI site. The foreign-gene transcription cassette include; PmeI restriction enzyme site sequence, 15 nt of NDV UTR, NDV gene end (GE) signal, one T nucleotide as intergenic sequence, NDV gene start (GS) signal, nucleotides for maintaining rule of six, Kozak sequence and ORF of the foreign gene. SEQ ID NO:1 is to the left of “ . . . ” and SEQ ID NO:2 is to the right of “ . . . ”.

FIG. 2 shows a Western blot analysis of rNDVs expressing S or S2 protein of IBV. The expression of codon optimized S, and S2 proteins and non-codon optimized S protein of IBV were detected by Western blot analysis in infected DF-1 cell lysates, using a chicken polyclonal anti IBV serum (A & B). For the codon optimized S protein of IBV expressed from rNDV (A-lane 3 and B-lane 2) two bands on top (˜170-220 kDa) represent uncleaved S protein (S0) or polymeric folded forms of S1 or S2 protein. The ˜130 kDa band, ˜95 kDa band and ˜60 kDa band represent S2 or S1 subunit of cleaved S protein. In the case of non-codon optimized S protein expressed from rNDV (2A-lane 2 and B-lane 1), two bands on top (˜170-220 kDa) represent uncleaved S protein (S0) or polymeric folded forms of S2 or S1 protein and ˜95 kDa band represents S2 or S1 subunit of cleaved S protein. In the case of rNDV/IBV-S2 (A-lane 1), there are two bands (˜170-220 kDa) on top, representing polymeric folded forms of S2 protein, the ˜105 kDa and the ˜95 kDa band representing S2 subunit. Lane 1 of panel A and lane 3 of panel B represent rNDV as control. The incorporation of codon optimized S2 and S proteins and non-codon optimized S protein of IBV in NDV particles were detected by Western blot. Two bands (˜170-220 kDa) on top represent uncleaved S protein (S0) or polymeric folded forms of S2 or S1 protein (C-lane 2). The ˜95 kDa and the ˜60 kDa band represent S2 or S1 subunit of cleaved S protein (C-lane 2). The two bands (˜170-220 kDa) on top represent polymeric folded forms of S2 protein, the ˜105 kDa band and the ˜95 kDa band represent S2 subunit (C-lane 4). The lane 1 and 3 of panel C represent purified rNDV control purified rNDV expressing non-codon optimized S protein, respectively. A monoclonal anti-NDV/HN antibody was used to detect the 70 kDa of HN protein of NDV in DF-1 cell lysates (A-lower panel); rNDV/S2 (lane 1), rNDV/IBV-non-cod.opt.S (lane 2), rNDV/IBV-cod.opt.S (lane 3), rNDV (lane 4) and incorporated in NDV particles (C-lower panel); rNDV (lane 1), rNDV/IBV-cod.opt.S (lane 2), rNDV/IBV-non-cod.opt.S (lane 3) and rNDV/S2 (lane 4).

FIG. 3 shows a Western blot analysis of rNDV expressing S1 protein of IBV. The expression of codon optimized S1 protein of IBV expressed from four individual rNDVs expressing four different expression cassettes of S1 protein were detected using Western blot in infected DF-1 cells infected with rNDVs, using a chicken polyclonal anti IBV serum. The lanes 1-5 represent cell lysates of rNDV, rNDV/S1, rNDV/S1+IBV-S-TM&CT, rNDV/S1(cs−)+NDV-F-TM&CT, rNDV/S1(cs+)+NDV-F-TM&CT in DF-1 cells and the lanes 8-12 represent infected DF-1 cell supernatants of rNDV, rNDV/S1, rNDV/S1+IBV-S-TM&CT, rNDV/S1 (cs−)+NDV-F-TM&CT, rNDV/S1 (cs+)+NDV-F-TM&CT, respectively. A ˜130 kD band represent expression of S1 protein by rNDV/S1+IBV-S-TM&CT, rNDV/S1(cs−)+NDV-F-TM&CT and rNDV/S1(cs+)+NDV-F-TM&CT in infected DF-1 cell lysate (lanes 3-5) and rNDV/S1 in infected DF-1 cell supernatant (lane 9).

FIG. 4 shows multicycle growth kinetics of the rNDV constructs in DF-1 cells. DF-1 cells were infected with each recombinant virus at a MOI of 0.01. Two hundred 11.1 of supernatant from infected cells were collected and replaced with fresh DMEM including 10% normal allantoic fluid at 8 h intervals. The titer of the virus in harvested samples were determined by TCID₅₀ assay in DF-1 cells.

FIG. 5 shows protective efficacy of rNDV constructs in one-day-old immunized SPF chickens against virulent IBV challenge (IBV protection experiment 1) at 21 days post-immunization. A) Respiratory clinical signs of IBV following challenge with a 10^{3d} EID₅₀ of virulent IBV strain Mass-41. The immunized chickens were challenged with a virulent IBV strain Mass-41. The severity scores of IBV clinical signs include; ocular discharge, nasal discharge and difficulty in breathing (0=normal to 3=heavy ocular discharge and heavy nasal discharge with mouth breathing) were recorded for 10 days after challenge. B) Relative viral load was determined by RT-qPCR in tracheal swab samples at day five following virulent IBV challenge. The relative viral load was expressed as mean reciprocal ±SEM log 10.

FIG. 6 shows protective efficacy of rNDV expressing codon optimized S protein of IBV against IBV challenge in immunized SPF chickens at 4-week-old age. A) Respiratory clinical signs of IBV following challenge with virulent IBV strain Mass-41. Three weeks after immunization, chickens were challenged with 10^{3.1} EID₅₀ of a virulent IBV strain Mass-41. The severity scores of IBV clinical signs include ocular discharge, nasal discharge and difficulty in breathing (0=normal to 3=heavy ocular discharge and heavy nasal discharge with mouth breathing) were recorded for 10 days after challenge. B) Relative viral load was determined by RT-qPCR in tracheal swab samples at day 5 following virulent IBV challenge. The relative viral load was expressed as mean reciprocal ±SEM log 10.

FIG. 7 shows protective efficacy of rNDV expressing codon optimized S protein of IBV and neutralizing antibody response against high dose of IBV challenge in immunized SPF chickens at 4-week-old age. A) Respiratory clinical signs of IBV following challenge with virulent IBV strain Mass-41. Three weeks after immunization, chickens were challenged with 10⁴⁷ EID₅₀ of a virulent IBV strain Mass-41. The severity scores of IBV clinical signs include ocular discharge, nasal discharge and difficulty in breathing (0=normal to 3=heavy ocular discharge and heavy nasal discharge with mouth breathing) were recorded for 8 days after challenge. B) The tracheal viral load was determined at day 4 following virulent IBV challenge. Each tracheal fluid was tested for IBV specific lesions on chicken embryo by inoculation (0.1 ml) of one 10-day-old embryonated SPF chicken egg. C) Neutralizing antibody response against IBV. Antibodies induced against IBV were assessed using virus neutralization assay. Serum titers are expressed as reciprocals Log 2 dilution.

FIG. 8 shows protective efficacy of rNDVs against virulent NDV challenge and antibody responses against NDV in SPF chickens immunized at 4-week-old. A) The protective efficacy of rNDV and rNDV expressing codon optimized S gene of IBV strain Mass-41 against highly virulent NDV strain Texas GB challenge. B) HI antibody titers against NDV were assessed using HI assay. Serum titers are expressed as reciprocals of Log 2 dilution.

### DETAILED DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

Ranges of values are disclosed herein. Unless otherwise stated, the ranges include all values to the magnitude of the smallest value (either lower limit value or upper limit value) and ranges between the values of the stated range.

The present disclosure relates to modified viral vectors to control infectious bronchitis (IB). Development of viral vectored vaccines to control IB according to this disclosure is not expected to lead to creation of variant viruses, which is a major drawback of current live attenuated IBV vaccines. The present disclosure demonstrates making and using representative modified vectors using Newcastle disease virus (NDV). NDV and several other viruses, such as herpesvirus, fowl pox virus and adenovirus have been evaluated as vaccine vectors for IBV¹⁹⁻²³. However, data presented herein unexpectedly demonstrate that although the S1 and S2 proteins of IBV are known to contain virus neutralizing epitopes, the presence of the entire S protein provides a strong protective immune response. Thus, the disclosure provides recombinant NDV (rNDV) vectors that allow for incorporation of intact IBV S protein into rNDV particles.

NDV is an attractive vaccine vector for IBV, because it can be used as a multivalent vaccine. Further, and without intending to be constrained by any particular theory, it is considered that a recombinant NDV vector IBV vaccine of this disclosure will eliminate the emergence of vaccine-derived IBV resulting from mutations and recombination as previously reported^{12,13}. The protective efficacy of NDV-vectored IBV vaccine can be enhanced by employing an appropriate regimen of prime-boost immunization strategy.

NDV belongs to the genus Avulavirus in the family Paramyxoviridae. Paramyxovirus pathogens include measles virus, mumps virus, human respiratory syncytial virus, and the zoonotic paramyxoviruses Nipah virus and Hendra virus. The genus Avulavirus includes at least 13 serotypes of Avian Avulaviruses (AAvV). All strains of NDV belong to Avulavirus-type 1(AAvV-1), but the other serotypes can also be used as modified or unmodified vaccine vectors. The disclosure includes use of other such modified or unmodified avulaviruses to achieve multivalent immunizations that include an immune response to IBV.

With respect to NDV, virulent NDV strains cause a fatal neurological disease in chickens. NDV strain LaSota has been used as a safe and effective live vaccine for more than 60 years²⁴. Recombinant NDV (rNDV) strain LaSota has been evaluated as a vaccine vector against several avian pathogens including IB V²⁵⁻²⁹. In one study, rNDV expressing S2 protein of IBV was found to induce partial protection against a virulent IBV challenge²². In another study, it was shown that a rNDV expressing S1 protein of IBV resulted in inducing protective immunity against virulent IBV challenge²³. However, the present disclosure demonstrates superiority of using an rNDV that includes larger segment of the intact S protein that at a minimum spans the S1/S2 cleavage site, as described further below.

The disclosure includes all polynucleotide and amino acid sequences described herein, and every polynucleotide sequence referred to herein includes its complementary DNA sequence, and also includes the RNA equivalents thereof to the extent an RNA sequence is not given. Every DNA and RNA sequence disclosed herein is encompassed by this disclosure, including but not limited to sequences encoding all viral and recombinant proteins that comprise a segment of an avian paramyxovirus protein and an infectious bronchitis virus S protein, as described further below. Where a DNA sequence is provided it may be a cDNA sequence of a viral negative sense genome; the skilled artisan can from the cDNA sequence readily envision the negative sense strand in its RNA form. The disclosure includes all negative strand viral genome sequences, and all complementary (cRNA) sequences thereof. In embodiments, the RNA sequences can comprise non-templated G residues arising from RNA editing. In embodiments, the disclosure includes viral particles that comprise an engineered paramyxovirus antigenome.

All of the amino acid sequences and nucleotide sequences associated with accession numbers disclosed herein are incorporated herein by reference as they exist in the database as of the effective date of the filing of this application or patent. Representative and non-limiting examples of complete NDV LaSota cDNA sequence and the amino acid sequences of proteins included in NDV viral particles are available under NCBI accession no. AF077761.1. The amino acid of the NDVfusion protein (F) of NDV strain LaSota (AFo77761.1) is also described below.

Thus, the amino acid and nucleotide sequences of a variety of strains of Newcastle and IBV viruses are known in the art, and it is contemplated that the segments of such proteins as described herein can be used and/or modified for use with embodiments of this disclosure. In certain embodiments, a protein or segment thereof of this disclosure may differ from a reference sequence. Thus, in certain examples the disclosure comprises a modified segment of a viral protein that comprises at least one amino acid change relative to the unmodified (wild type) counterpart. In certain examples more than one amino acid change can be included. Such changes can comprise conservative or non-conservative amino acid substitutions, insertions, and deletions, provided the modified sequence retains or improves on the capability to be used to stimulate an immune response. In embodiments an NDV fusion (F), M or P amino acid sequence, or a combination thereof, as used herein is at least 80%-99% similar to a reference sequence. Likewise, in embodiments, an IBV S amino acid sequence used herein is at least 80%-99% similar to a reference sequence. As will be apparent from the description herein and figures of this disclosure, in embodiments, the viral genome comprises the NDV NP, P, M, F, HN and L genes, and include the IBV S gene inserted between the P and M proteins, although other insertion sites are contemplated.

In embodiment, the S protein that is incorporated into the rNDVs of this disclosure comprises a contiguous segment of IBV S protein that spans an IBV cleavage site between IBV S1 and IBV S2 proteins. In one embodiment, the amino acid sequences of an IBV S1 proteins comprises a furin consensus motif of RRFRR (AY851295.1; SEQ ID NO:3 for infectious bronchitis virus strain Mass 41, as present in the spike glycoprotein). In an embodiment the cleavage site comprises RRFRRS SEQ ID NO:4). The contiguous segment of IBV S protein that contains the S1/S2 cleavage site is of an adequate length such that sufficient epitopes of the S protein that can elicit a protective immune response against IBV are included. In embodiments, the segment comprises between 500-1000 amino acids of the S protein, inclusive, including all integers and ranges of integers there between. In embodiments, the segment comprises amino acids 532-538 of representative IBV protein provided in the spike glycoprotein under AY851295.1 within a contiguous segment of between 500-1000 amino acids of AY851295.1. In embodiments, the S protein component of the N-terminal domain (rNTDs) of this disclosure have at from 80%-100% sequence identity to any such segment of AY851295.1. In embodiments, the S protein component of the rNTDs of this disclosure has at least 95% identity across the entire length of the S protein amino acid sequence of spike glycoprotein under AY851295.1 In certain non-limiting embodiments, the disclosure includes using one or more of the NTD proteins or the IBV S protein as a chimeric protein that further comprises at least a second polypeptide sequence from a pathogen that is not NDV or IBV, thereby forming at least a multivalent immunogenic agent, and wherein the second polypeptide stimulates a protective immune response to the pathogen that is not NDV or IBV. Non-limiting examples of sources of the second polypeptide (or more than a second polypeptide, such as a third or fourth polypeptide) include antigens from any avian pathogen, including internal and external pathogens. Thus, in embodiments, the second polypeptide can be derived from any non-NDV and non-IBV virus or other pathogenic agent that causes any disease of poultry, including by not limited to: viral inclusion body hepatitis, haemorrhagic enteritis of turkeys, egg drop syndrome, adenovirus group 1—associated infections, infectious bursal disease (gumboro), laryngotracheitis, swollen head syndrome, infectious encephalomyelitis, leucosis, Marek's disease, and fowl pox and reovirus infections. Specific pathogens that can be a source of the second polypeptide include Escherichia coli, Salmonella Pullorum/Gallinarum, Pasteurella multocida, Avibacterium paragallinarum, Gallibacterium anatis, Ornitobacterium rhinotracheale, Bordetella avium, Clostridium perfringens, Mycoplasma spp., Erysipelothrix rhusiopathiae, and Riemerella anatipestifer. In embodiments, the second polypeptide is present in a chimeric protein that includes the IBV S protein, or is present in an NDV protein. In embodiments, the second protein is a complete protein from a non-IBV and non-NDV protein, or is an immunogenic fragment of such a protein. In embodiments, the second polypeptide is a component a foreign protein is from 10-500 amino acids in length, inclusive, and including all integers and ranges of integers there between.

To produce the viral particles, the viral particles themselves, or DNA/cDNA or RNA or cRNA encoding the required set of proteins can be introduced directly into producer cells, and shed viral particles can be isolated from the cells. In embodiments, one or more expression vectors can be used to produce the viral particles. In this regard, a variety of suitable expression vectors known in the art can be adapted to produce the modified paramyxovirus particles of this disclosure. In general, the expression vector comprises sequences that are operatively linked with the sequences encoding the viral particle proteins that comprise an IBV S gene between the NDV P and M genes, or another suitable insertion site. A particular polynucleotide sequences is operatively-linked when it is placed in a functional relationship with another polynucleotide sequence. For instance, a promoter is operatively-linked to a coding sequence if the promoter affects transcription or expression of the coding sequence. Generally, operatively-linked means that the linked sequences are contiguous and, where necessary to join two protein coding regions, both contiguous and in reading frame. However, it is well known that certain genetic elements, such as enhancers, may be operatively-linked even at a distance, i.e., even if not contiguous, and may even be provided in trans. Promoters present in expression vectors that are used in the present disclosure may be endogenous or heterologous to the host cells, and may be constitutive or inducible. Expression vectors can also include other elements that are known to those skilled in the art for propagation, such as transcription and translational initiation regulatory sequences operatively-linked to the polypeptide encoding segment. Suitable expression vectors may include, for example, an origin of replication or autonomously replicating sequence (ARS) and expression control sequences, an enhancer and other regulatory and/or functional elements, such as ribosome-binding sites, RNA splice sites, polyadenylation sites, transcriptional terminator sequences, and mRNA stabilizing sequences, as well as a wide variety of selectable markers.

In certain embodiments, the viral particles can be produced using a set of plasmids, which may be used in conjunction with a cDNA, such as an antigenome of an NDV that is modified to include a polynucleotide sequence that encodes (or can be transcribed to encode) an IBV S protein, the coding sequence for which can be placed between the NDV P and M genes. In embodiments, a full length cDNA of NDV can be co-transfected into suitable cells with one or more plasmids that express, for example, the N, P and L genes of NDV so that recombinant NDV particles can be produced by the cells, and recovered. In this regard, the expression vectors can be introduced into the host producer cells by any method known in the art. These methods vary depending upon the type of cellular host, and include but are not limited to transfection employing cationic liposomes, electroporation, calcium chloride, rubidium chloride, calcium phosphate, DEAE-dextran, or other substances as will be apparent to the skilled artisan. In embodiments the viral particles are produced using chicken embryo fibroblast cells, such as DF-1 cells, or mammalian cells, such as HEp-2 cells. In embodiments, the rNDVs are produced in embryonated, specific-pathogen-free (SPF) eggs. Methods of making the modified virus particles are accordingly included, and generally comprise introducing polynucleotides encoding the viral genome and viral proteins virus into suitable producer cells, and recovering shed virus from them. Thus, cells and cell cultures that harbor polynucleotides encoding the modified paramyxoviruses of this disclosure are included, as are isolated and/or purified modified paramyxovirus viral particle preparations. The particles can be purified to any desired degree of purity using standard approaches, such as density gradient separation or commercially available kits used to purify enveloped viruses or exosomes.

In certain embodiments, the disclosure provides a modified antigenomic RNA of Newcastle disease virus, comprising or consisting of: NDV NP gene, NDV P gene, IBV S gene, NDV M gene, NDV F gene, NDV HN gene and NDV L gene, which can be in this order from a 3′ to 5′ direction. In embodiments, the disclosure includes making such modified RNAs and rNDV particles by adapting approaches described in U.S. Pat. Nos. 9,476,033 and 7,244,558, the entire disclosures of which are incorporated herein by reference. In embodiments, the disclosure includes making and using a cDNA encoding all or a segment of the antigenome of any rNDV disclosed herein.

In certain aspects the disclosure includes a pharmaceutical formulation comprising modified paramyxovirus particles as described herein. The form of pharmaceutical preparation is not particularly limited, but generally comprises modified viral particles and at least one inactive ingredient. In certain embodiments suitable pharmaceutical compositions can be prepared by mixing any one type of the particles, or combination of distinct types of particles, with a pharmaceutically-acceptable carrier, diluent or excipient, or immune response regulator, or an antibiotic, and suitable such components are well known in the art. Some examples of such carriers, diluents and excipients can be found in: Remington: The Science and Practice of Pharmacy (2005) 21st Edition, Philadelphia, Pa. Lippincott Williams & Wilkins, the disclosure of which is incorporated herein by reference. In embodiments, a vaccine formulation is provided. In embodiments, a formulation of this disclosure is provided as an effervescent tablet, a pellet, or in a lyophilized form. The pharmaceutical composition can be also include, for example, any suitable adjuvant.

In embodiments, the rNDV particles are administered to avian animals using any suitable route. In embodiments, the particles are provided as vaccines and can be administered orally, intranasally, intraocularly or parenterally, e.g. by intramuscular or subcutaneous injection. In embodiments, the vaccine formulations are administered via an oculanasal route, thus conjunctival and intranasal routes are included. In embodiments, the vaccine is administered in drinking water, or as an aerosol or a spray. In embodiments, a vaccine formulation of this disclosure is administered in ovo, as an eye drop, or injection, such as a subcutaneous or wing-web injection. In embodiments, the disclosure includes an article of manufacture, such as a kit, the kit comprising rNTDs as described herein in any suitable form, wherein the rNTDs are comprised within one or more containers, and wherein the article or kit comprises printed material, such as a label or insert that includes instructions for using the rNTDs for vaccination of avian animals.

In embodiments, immunologically effective amount rNDV particles are administered. Immunologically effective as used herein means an amount that results in production of neutralizing antibodies against IBV, and/or an results in an improved severity score for respiratory clinical signs of IBV infection, and/or results in reduced shedding of the challenge virus, i.e., shedding of an infectious IBV virus, the avian animal encounters in its environment. An amount of shedding can be determined according to numerous approaches known to those skilled in the art. Likewise, respiratory clinical signs of IBV infection and scoring systems for quantitating or quantifying the clinical signs are known. Such signs include but are not necessarily limited to: young avian animals, such as chickens, are depressed and huddle under a heat source; respiratory signs that include but are not limited to gasping, coughing, tracheal rales and nasal and ocular discharge; birds in lay exhibit a marked drop in egg production and an increased number of poor quality eggs may be produced; the external and internal quality of the eggs can be affected and result in soft-shelled or misshapen eggs misshapen, and/or eggs with an abnormal water content; hatchability rate of the eggs can also be affected. In an embodiment the disclosure comprises determining a severity score of clinical signs of IBV infection that include nasal discharge, ocular discharge and difficulty in breathing. In embodiment, the scoring system comprises the following values: 0=normal, to 3=heavy ocular discharge and heavy nasal discharge with mouth breathing. When avian kidneys are affected, increased water intake, depression, scouring and wet litter may be observed. Any measure of immunologic efficacy of this disclosure can be compared to any suitable reference, examples of which include but are not limited a standardized curve, a titration, an area under a curve, or a comparison to any matched or otherwise suitable control.

In embodiments, an immunologically effective amount results in preventing and/or lessening of clinical disease and/or mortality when challenged with a virulent NDV. Immunological protection can be durable, and last for days, weeks or months, or longer, after vaccination, and such vaccinations can be effective to elicit such protection after a single dose, or multiple doses. Booster vaccinations can be used according to schedules that are known in the art and can be adapted for use with vaccines of this disclosure when provided the benefit of this specification, and include such approaches as a Prime-Boost strategy.

In embodiments, neutralizing antibodies are produced. The term “neutralizing antibody” refers to an antibody or a plurality of antibodies that inhibits, reduces or completely prevents viral infection. Whether neutralizing antibodies are produced can be determined by in vitro assays that are known in the art.

In embodiments, viral load in the vaccinated animals is reduced. Viral load can be determined according to methods known to those skilled in the art, including but not limited to observation of IBV specific lesions on chicken embryo, and PCR amplifications, including but not limited to quantitative real-time polymerase chain reaction assays. In embodiments, viral RNA is determined from a tracheal swab sample.

In embodiments, an embryo infective dose (EID) is used. In embodiments, a dosage of mean embryo infectious vaccine dose (EID50) of 104-106 is used. In embodiments, a dose of at least 106 EID50 is used. In embodiments, any suitable multiplicity of infection (MOI) can be used.

In embodiments, an rNDV is modified such that it is less pathogenic than an un-modified NDV, and thus may comprise or be derived from an avirulent rNDV, or it may be provided as an attenuated or inactivated vaccine. In embodiments, an rNDV for use in vaccines of this disclosure are derived from NDV strains from the class II genotype I (i.e. 12, V4, and PHY-LMV42). In embodiments, an rNDV that is resistant to heat is used. In embodiments, the rNDV is derived from a LaSota, B1, or VG/GA type NDV.

In embodiments, the avian animals to which compositions of this disclosure are administered are any type of poultry. In embodiments, the avian animals are Galliformes and thus include any members of the order of heavy-bodied ground-feeding birds that includes turkey, grouse, chicken, New World quail and Old World quail, ptarmigan, partridge, pheasant, junglefowl and the Cracidae. In embodiments, the avian animals are domesticated fowl, including but not limited to domesticated chickens and turkeys. In embodiments, the chickens are Gallus gallus, such as Gallus gallus domesticus. In embodiments, the chickens are roosters or hens. In embodiments, the avian animals are adults, juveniles, or embryos. In an embodiment, a composition of this disclosure is applied to eggs. In embodiments, vaccines of this disclosure administered to a population of avian animals, i.e., a flock. In embodiments, from 50-85% or more members of the flock are vaccinated to achieve, for example, herd or flock immunity.

The Examples that follow demonstrate generation of recombinant rNDVs expressing the S1, S2 and S proteins of IBV using reverse genetics technology. The results showed that the rNDV expressing the S protein of IBV provided better protection than the rNDV expressing S1 or S2 protein of IBV, indicating that the S protein is the better protective antigen of IBV. Immunization of 4-week-old SPF chickens with the rNDV expressing S protein elicited IBV-specific neutralizing antibodies and provided complete protection against virulent IBV and virulent NDV challenges. These results indicate that the rNDV expressing the S protein of IBV is a safe and effective bivalent vaccine candidate for both IBV and NDV. The Examples are presented to illustrate the present disclosure. They are not intended to limiting in any matter.

### Example 1

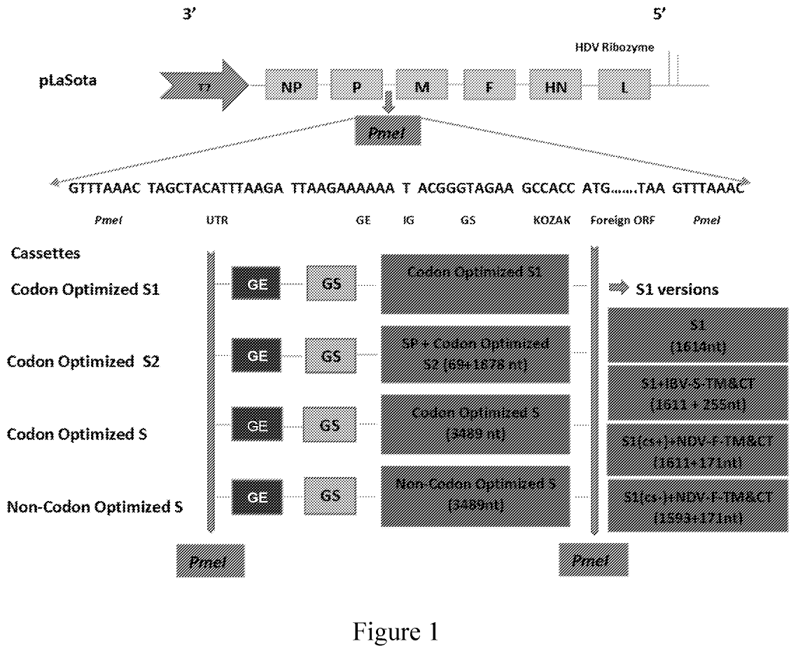

Generation of rNDVs expressing S1, S2 and S protein of IBV. The expression cassettes containing the codon optimized S1, S2, S and non-codon optimized S genes of IBV were cloned into the cDNA encoding the complete antigenome of NDV strain LaSota, using the PmeI site, between P and M genes (FIG. 1). The correct sequences of genes cloned into full length cDNA of NDV were confirmed by nucleotide sequence analysis. Each full length cDNA was co-transfected with three expression plasmids containing N, P or L gene of NDV strain LaSota into MVA-T7 infected HEp-2 cells. Infectious recombinant NDVs containing S1, S2 and S genes of IBV were recovered from all cDNAs. The sequences of S1, S2 and S genes present in the rNDVs were confirmed by sequence analysis of RT-PCR products. To evaluate genetic stability of rNDVs, the viruses were passaged five times in 9-day-old embryonated SPF chicken eggs. The nucleotide sequence analysis of the S1, S2, and S genes showed that the inserted ORFs were maintained without any adventitious mutations.

Evaluation of the expression of the S1, S2 or S protein of IBV.

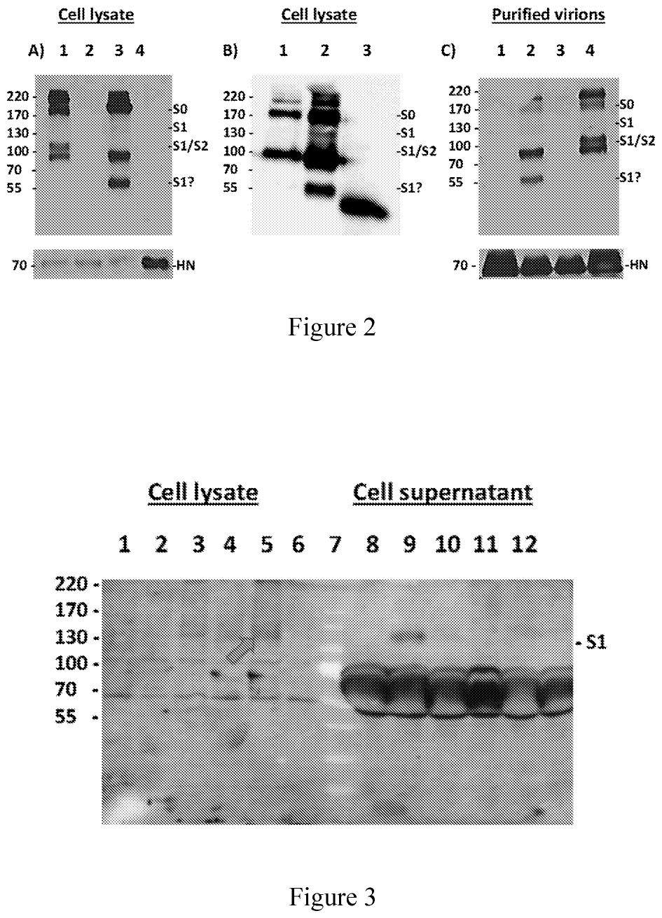

The expression of codon optimized S2, and S proteins and non-codon optimized S protein of IBV strain Mass-41 by rNDV constructs was detected by Western blot analysis of DF-1 using a chicken polyclonal anti IBV serum (FIGS. 2A & B). The expression level of codon optimized S protein of IBV was significantly higher than that of the non-codon optimized S protein of IBV. For the codon optimized S protein of IBV expressed from rNDV (FIG. 2A—Lane 3 and FIG. 2B—lane 2), the two bands on top (˜170-220 kDa) probably represent either uncleaved S protein (S0) or polymeric forms of S protein. The ˜130 kDa, the ˜95 kDa and the ˜60 kDa band represent S1 or S2 subunit of cleaved S protein of IBV. In the case of non-codon optimized S protein of IBV expressed from rNDV (FIG. 2A-lane 2 and FIG. 2B-lane 1), the two bands on top (˜170-220 kDa) probably represent uncleaved S protein (S0) or polymeric forms of S protein. The ˜95 kD band represents S2 or S1 subunit of cleaved S protein of IBV. In the case of rNDV/IBV-S2 strain (FIG. 2A-lane 1), there are two bands (˜170-220 kDa) on top, representing polymeric folded forms of S2 protein, a ˜105 kDa band and a ˜95 kDa band representing S2 subunit. Lane 4 of FIG. 2A and lane 3 of FIG. 2B represent rNDV as control. These results showed that codon optimized S and S2 proteins of IBV were expressed efficiently. The non-codon optimized S protein was also expressed from rNDV, but not efficiently and not consistently. A monoclonal anti-NDV/HN antibody was used to detect a 70 kDa of HN protein of NDV in lysates, confirming similar level of NDV protein in each lane (FIG. 2A—lower panel). We further evaluated incorporation of IBV S and S2 proteins into NDV virions. The rNDVs expressing codon optimized S and S2 proteins and rNDV expressing non-codon optimized S protein were inoculated into eggs, 3 days after inoculation, viral particles in infected allantoic fluid were partially purified. Two bands (˜170-220 kDa) on top, representing S protein, a ˜95 kDa band and a ˜60 kDa band representing S2 or S1 subunit of cleaved S protein, were detected in purified particles of rNDV expressing codon optimized S protein by Western blot analysis (FIG. 2C lane 2). The lane 4 of FIG. 2C shows two bands (˜170-220 kDa) on top, representing polymeric folded of S2 protein, a ˜105 kDa band and a ˜95 kDa band representing S2 subunit. The lane 1 of FIG. 2C represents purified rNDV control and lane 3 of FIG. 2C shows purified rNDV expressing non-codon optimized S protein. These results suggested that the codon optimized S and S2 proteins of IBV expressed by rNDVs were incorporated into rNDV particles. A monoclonal anti-NDV/HN antibody was used to detect a 70 kDa of HN protein of NDV in partially purified virions, confirming similar level of NDV protein in each lane (FIG. 2C—lower panel).

The expression of codon optimized S1 protein expressed from four individual rNDV constructs were detected by Western blot analysis in lysates of infected DF-1 cells, using a chicken polyclonal anti IBV serum (FIG. 3). The lanes 1-5 represent infected DF-1 cell lysates of rNDV, rNDV/S1, rNDV/S1+IBV-S-TM&CT, rNDV/S1(cs−)+NDV-F-TM&CT, rNDV/S1(cs+)+NDV-F-TM&CT. The lanes 8-12 represent infected DF-1 cell supernatants of rNDV, rNDV/S1, rNDV/S1+IBV-S-TM&CT, rNDV/S1(cs−)+NDV-F-TM&CT, rNDV/S1(cs+)+NDV-F-TM&CT in DF-1 cells, respectively. A ˜130 kDa band representing expression of S1 by rNDV/S1+IBV-S-TM&CT, rNDV/S1(CS−)+NDV-F-TM&CT, and rNDV/S1(cs+)+NDV-F-TM&CT in lysate of DF-1 cells (FIG. 3 lanes 3-5) and rNDV/S1 in infected DF-1 cell supernatant (lane 9) was observed. Our results showed that the S1 protein was expressed at very low level by all the rNDVs based on Western blot analysis. Only the unmodified S1 protein was detected in the cell culture supernatant.

Growth Characteristics of rNDV Constructs.

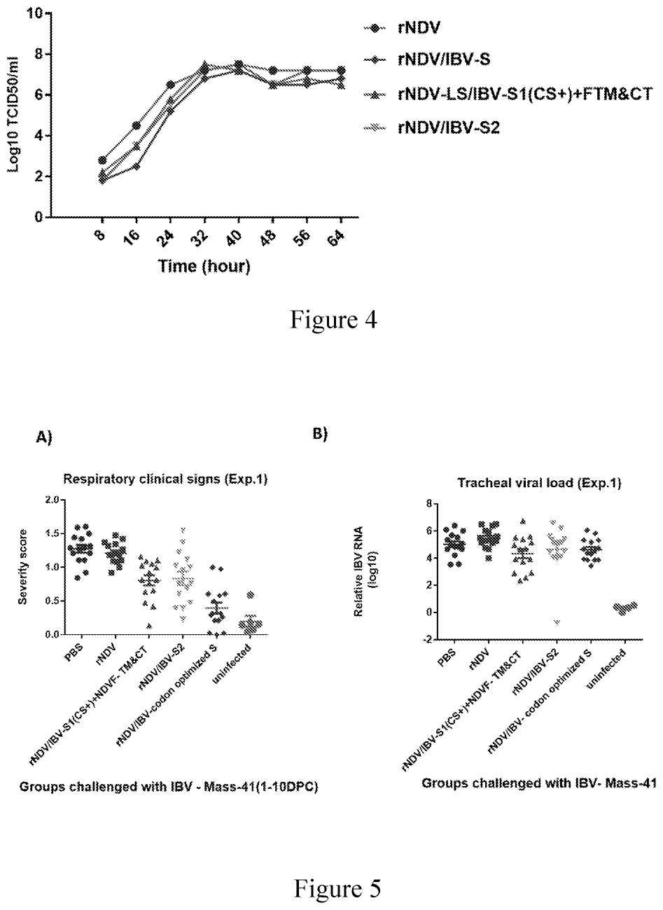

The recovered rNDVs were passaged four times in 9-day-old embryonated SPF chicken eggs. All the viruses were able to replicate well in eggs (>2⁸ HAU/ml). The multicycle growth kinetics of rNDV/S1 (cs+)+NDV-F-TM&CT, rNDV expressing codon optimized S protein of IBV and empty rNDV vector were evaluated in the presence of exogenous protease in DF-1 cells (FIG. 4). Compared to the parental virus, rNDV expressing codon optimized S protein of IBV grew slightly less efficiently. The maximum titer of parental virus reached 10^{7.5} TCID₅₀/ml at 40 hours post infection, whereas the maximum titer of rNDV expressing codon optimized S gene of IBV reached 10^{7.2} TCID₅₀/ml at 40 hours post infection. These results indicated that presence of S gene did not significantly affect the growth characteristics of rNDV.

The protective efficacy of rNDVs expressing S1, S2 or S protein of IBV in chickens against a virulent IBV challenge. IBV protection experiment 1.

To evaluate the protective efficacy of rNDVs expressing S1, S2 or S protein of IBV, SPF chicks were immunized at 1-day-old age with each virus via oculanasal route. At three weeks post-immunization, chickens were challenged with virulent IBV strain Mass-41. The severity scores of IBV clinical signs were recorded twice daily for 10 days post-challenge (FIG. 5-A). Compared to chickens immunized with parental rNDV and chickens inoculated with PBS, chickens immunized with rNDVs expressing codon optimized S, S1 or S2 protein of IBV showed significantly less severe of clinical signs (P<0.05). Among groups of chickens immunized with rNDVs expressing codon optimized S1, S2 or S protein, the group immunized with rNDV expressing codon optimized S protein showed the least severity of clinical signs (P<0.05). In order to evaluate the efficacy of rNDVs expressing S1, S2 or S protein of IBV in preventing shedding of virulent IBV challenge virus in immunized chickens, on day five post-challenge, tracheal swab samples were collected from five birds of each group and were evaluated for the viral load by RT-qPCR. Our results did not show significant difference in virus shedding among groups of immunized chickens at day five post challenge (FIG. 5B).

IBV protection experiment 2.

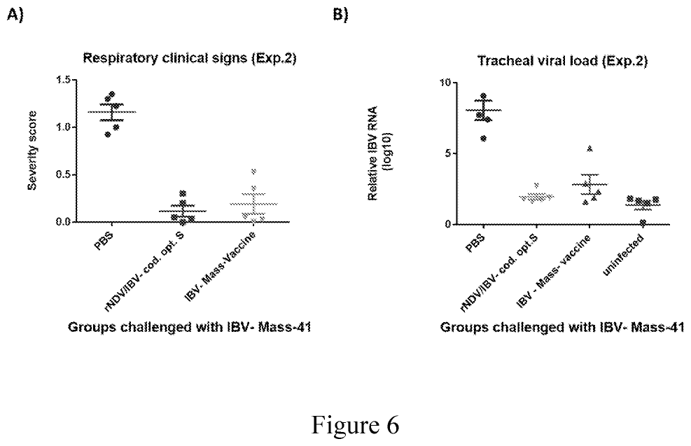

To evaluate the protective efficacy of rNDVs expressing codon optimized S protein of IBV in adult chickens, SPF chickens were immunized at 4-week-old age. The protective efficacy of rNDV expressing codon optimized S gene of IBV was determined by challenging the immunized chickens with OIE recommended dose (10³¹ EID₅₀) of virulent IBV strain Mass-41 at 3 week post-immunization. The severity scores of IBV clinical signs were recorded twice a day for 10 days post-challenge (FIG. 6A). Compared to chickens inoculated with PBS, chickens immunized with rNDV expressing codon optimized S protein of IBV and chickens immunized with a commercial live attenuated IBV vaccine showed significantly less severe clinical signs (P<0.05). In order to evaluate the efficacy of rNDV expressing S protein of IBV in preventing shedding of virulent IBV in immunized chickens, at day 5 following challenge with a virulent IBV, the tracheal swab samples collected from five chickens of each group were analyzed for the viral load by RT-qPCR. Our results showed that chickens vaccinated with rNDV expressing codon optimized S protein of IBV and chickens vaccinated with a commercial IBV vaccine showed very low levels of viral load in the trachea, whereas chickens inoculated with PBS showed high levels of viral load in the trachea (P<0.05) (FIG. 6B).

IBV protection experiment 3.

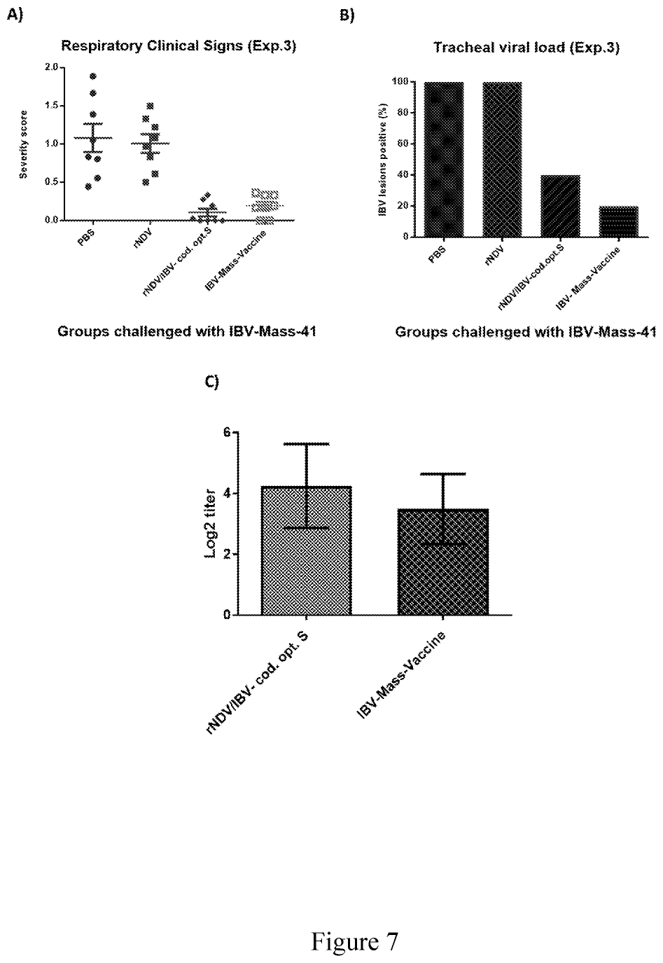

To evaluate the protective efficacy of rNDVs expressing codon optimized S protein of IBV in adult chickens against a higher dose of virulent IBV challenge, SPF chickens were immunized at 4-week-old age. The protective efficacy of rNDV expressing codon optimized S gene of IBV was determined by challenging the immunized chickens with 10⁴⁷ EID₅₀ virulent IBV strain Mass-41 at 3 week post-immunization. The severity scores of IBV clinical signs were recorded twice a day for 8 days post-challenge (FIG. 7A). Compared to chickens inoculated with PBS, chickens immunized with rNDV expressing codon optimized S protein of IBV and chickens immunized with a commercial live attenuated IBV vaccine showed significantly less severe clinical signs (P<0.05). In order to evaluate the efficacy of rNDV expressing S protein of IBV in preventing shedding of virulent IBV in immunized chickens, at days 4 following challenge with virulent IBV, the tracheal swab samples collected from five chickens of each group were assayed for the IBV specific lesions in chicken embryo. Our results showed that 2 out of 5 (40%) chickens vaccinated with rNDV expressing codon optimized S protein of IBV and 1 out of 5 (20%) chickens vaccinated with a commercial IBV vaccine were shedding virus in trachea, respectively, whereas 5 out of 5 (100%) of chickens immunized with parental rNDV and 5 out of 5 (100%) of chickens inoculated with PBS were shedding virus in the trachea (P<0.05) (FIG. 7B).

The protective efficacy of rNDVs against a highly virulent NDV challenge.

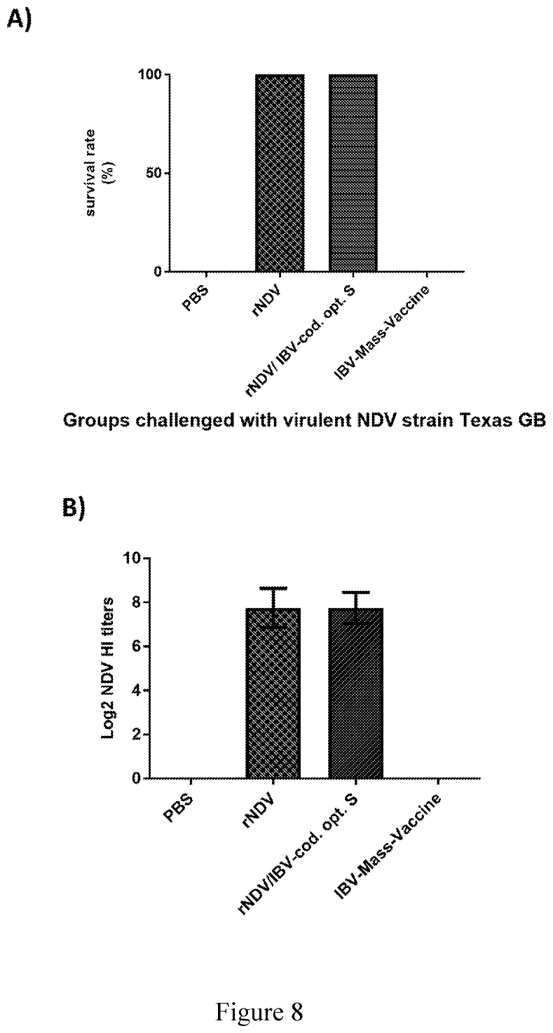

To evaluate the protective efficacy of rNDV expressing S gene of IBV against a virulent NDV strain, groups of five 4-week-old chickens were immunized with rNDV, rNDV expressing codon optimized S protein, commercial IBV vaccine and PBS. Three weeks after immunization, chickens were challenged with virulent NDV strain Texas GB in our BSL-3 plus facility. Our results showed that chickens immunized with the rNDV and rNDV expressing codon optimized S gene of IBV were fully protected from highly virulent NDV challenge, while all chickens in commercial IBV vaccine and PBS groups died or showed clinical signs of virulent NDV infection (FIG. 8A).

Antibodies produced against IBV and NDV.

Hemagglutination inhibition (HI) assay using a standard protocol of OIE was used to assess the level of antibodies mounted against NDV in serum samples of chickens 21 days after immunization. HI titers of NDV was detected in serum samples of all chickens. There was no significant differences observed among HI titers against NDV in serum samples of chickens from groups immunized with rNDV and rNDV expressing S protein (FIG. 8B). Virus neutralization assay was performed according to a standard protocol of OIE to assess the level of neutralizing antibodies mounted against IBV strain Mass-41 in serum samples of chickens at 21 days after immunization. The results showed that the neutralizing antibodies against IBV were detected in serum samples of chickens immunized with rNDV expressing codon optimized S protein of IBV and with commercial live attenuated IBV vaccine (FIG. 7C). Neutralizing antibodies against IBV were not detected in 1:8 dilution of a serum sample from a chicken immunized with empty rNDV vector. This result showed that the rNDV expressing codon optimized S protein of IBV induces neutralizing antibodies against IBV.

It will be apparent from the foregoing that this disclosure provides a comparison of the protective efficacies of S1, S2 and S proteins of IBV using rNDV as a vaccine vector. The S1, S2 and S genes of IBV strain Mass-41 were individually inserted between the P and M genes of rNDV strain LaSota. Four different versions of IBV S1 gene were used to identify the version that is expressed at the highest level and incorporated into NDV particles. We were able to recover all the recombinant viruses and their growth characteristics were similar to rLaSota. The recombinant viruses containing IBV S genes grew slightly slowly than their parental virus. The viruses were genetically stable after five passages in SPF chicken embryos. Western blot analysis showed that chicken codon optimized S2 and S proteins were expressed at much higher levels and were incorporated into NDV particles. Whereas, all the four versions of S1 protein were detected at very low levels by Western blot analysis. It is noteworthy that the unmodified S1 protein was detected in the infected cell culture supernatant, indicating that the modification of S1 protein probably caused retention of the protein in the cell. These results suggest that the S2 protein acts as a chaperone to assist in the folding of the S1 protein. The S1 protein is folded incorrectly in the absence of S2 protein and the new structure causes loss of some conformational epitopes for IBV antibodies.

In the first IBV protection experiment, we found that 1-day-old chicks immunized with rNDV expressing the S protein of IBV conferred better protection from disease compared to 1-day-old chicks immunized with rNDVs expressing either S1 or S2 protein of IBV. Our results showed that the S protein, which contains both S1 and S2 proteins, is the best protective antigen of IBV. The S2 protein lacks major neutralizing epitopes which are present in the S1 protein, hence it is not an effective antigen. The S1 protein contains major neutralizing epitopes, but it losses some conformational epitopes when expressed separately. In this disclosure we showed that rNDV expressing S protein provided enhanced protection.

In the second IBV protection experiment, we investigated whether age of immunization influences the outcome of IBV challenge. Our results showed that a single immunization of 4-week-old chickens with rNDV expressing S protein completely protected chickens against IBV challenge based on disease and viral load in tracheas. Indeed, the level of protection conferred by rNDV expressing S protein was similar to that of a commercial IBV vaccine. This showed that protection was greater when the chickens were immunized at an age when their immune system is relatively well developed.

In the third IBV protection experiment, we showed that rNDV expressing IBV S protein protects adult chickens against a higher dose of virulent IBV challenge. However, compared to standard challenge dose of virulent IBV, a higher challenge dose of virulent IBV caused mild disease and tracheal viral load in adult chickens immunized with either rNDV expressing IBV S protein or commercial live attenuated IBV vaccine. Our result showed that although both the age of immunization and dose of challenge virus affect the results of IBV challenge, the influence of the age of immunization is greater than the effect of the dose of challenge virus.

### Example 2

The steps of the method described in the various embodiments and examples disclosed herein are sufficient to carry out the methods of the present invention. Thus, in embodiments, the method comprises or consists essentially of a combination of the steps of the methods disclosed herein. In another embodiment, the method consists of such steps.

Cells and viruses.

Chicken embryo fibroblast (DF-1) cells and human epidermoid carcinoma (HEp-2) cells were obtained from the American Type Culture Collection (ATCC, Manassas, Va.). They were grown in Dulbecco's minimal essential medium (DMEM) containing 10% fetal bovine serum (FBS). The recombinant avirulent NDV strain LaSota was generated previously in our lab using reverse genetics³⁰. The rNDV and rNDVs expressing chicken codon optimized S1, S2 and S genes and non-codon optimized S gene of IBV strain Mass-41 were grown in 9-day-old embryonated SPF chicken eggs at 37° C. The virulent IBV strain Mass-41 was propagated in 10-day-old SPF embryonated chicken eggs and harvested five days after infection. The titer of virus in harvested allantoic fluid was determined by 50% embryo infectious dose (EID₅₀) method. Briefly, ten-fold serial dilutions of IBV strain Mass-41 was inoculated into 10-day-old embryonated SPF chicken eggs. Seven days after inoculation, infected embryos were examined for IBV specific lesions such as stunting or curling. The titer of virus was calculated using Reed and Muench method³¹. The modified vaccinia virus strain Ankara expressing T7 RNA polymerase (MVA-T7) was propagated in monolayer primary chicken embryo fibroblast cells.

Generation of rNDVs containing S1, S2 or S gene of IBV.

A plasmid containing full-length antigenomic cDNA of NDV strain LaSota has been constructed previously³⁰. In this study, open reading frames (ORFs) of chicken codon optimized of S2 and S genes, non-codon optimized S gene and four versions of codon optimized S1 gene of IBV strain Mass-41 were constructed in seven individual transcription cassettes. The cassettes contained the following IBV genes; i) codon optimized S1 subunit of S gene (1611 nt), ii) codon optimized S1 gene (1611 nt) fused with N-terminus of transmembrane and cytoplasmic tail of S gene (255 nt), iii) codon optimized S1 subunit of S gene containing S1 protein cleavage site residues (1611 nt) fused with N-terminus of transmembrane and cytoplasmic tail of NDV F gene (171 nt), iv) codon optimized S1 subunit of S gene without S1 protein cleavage site residues (1593 nt) fused with N-terminus of transmembrane and cytoplasmic tail of NDV F gene (171 nt), v) codon optimized S2 subunit of S gene (1878 nt) of IBV fused with C-terminus of signal peptide sequence of S gene (69 nt), vi) codon optimized S gene (3489 nt) and vii) non-codon optimized S gene (3489 nt).

The transcription cassettes were modified to contain PmeI restriction enzyme sequence, 15 nt of untranslated region (UTR) of NDV, NDV gene end (GE) signal, one T nucleotide as intergenic sequence, NDV gene start (GS) signal, extra nucleotides to maintain the rule of six (19, 27), Kozak sequence at the upstream of foreign gene ORFs and PmeI restriction enzyme sequence at downstream of foreign gene ORF. The transcription cassettes were amplified from two plasmids containing commercially synthesized codon optimized and non-codon optimized S genes of IBV strain Mass-41 and cloned into complete antigenomic cDNA of rLaSota at P and M gene junction using PmeI site (FIG. 1). rNDVs containing the IBV genes were recovered by reverse genetics as described previously³¹. rNDVs containing S1 gene, S1 gene fused with transmembrane and cytoplasmic tail of S gene, S1 gene containing cleavage site residues of S gene of IBV fused with transmembrane and cytoplasmic tail of NDV F gene, S1 gene without cleavage site residues of S gene fused with transmembrane and cytoplasmic tail of NDV F gene, S2 gene, codon optimized S gene and non-codon optimized S gene were named rNDV/S1, rNDV/S1+IBV-S-TM&CT, rNDV/S1(cs+)+NDV-F-TM&CT, rNDV/S1(cs−)+NDV-F-TM&CT, rNDV/S2, rNDV/codon optimized-S and rNDV/non-codon optimized-S, respectively. The foreign genes were amplified from the rNDV/IBVs by RT-PCR. The correct sequences of the foreign genes were confirmed by nucleotide sequence analysis.

Expression of S1, S2 and S proteins of IBV.

Confluent monolayers of DF-1 cells were infected at a multiplicity of infection (MOI) of 0.01 with rNDV strain LaSota, rNDV/S1, rNDV/S1+IBV-S-TM&CT, rNDV/S1(cs+)+NDV-F-TM&CT, rNDV/S1(cs−)+NDV-F-TM&CT, rNDV/S2, rNDV/codon optimized-S or rNDV/non-codon optimized-S. DF-1 cells were harvested 30 hours post-infection, lysed and analyzed by Western blot. A standard polyclonal chicken anti-IBV serum was used to detect the expression of S1, S2 and S proteins of IBV. To determine the incorporation of IBV proteins into NDV envelope, rNDV, rNDV/S2, rNDV/codon optimized-S and rNDV/non-codon optimized-S were inoculated into 9-day-old embryonated SPF chicken eggs. Three days after incubation, recombinant viral particles from infected allantoic fluids were partially purified by sucrose density gradient centrifugation and analyzed by Western blot analysis.

Growth characteristics of rNDV constructs.

In order to determine the growth kinetics of rNDVs expressing S1, S2 or S protein of IBV, confluent monolayers of DF-1 cells in 6-well tissue culture plates were infected at a MOI of 0.01 with rNDV, rNDV/S1(cs+)+NDV-F-TM&CT, rNDV/S2 or rNDV/codon optimized-S and adsorbed for 90 minutes at 37° C. After adsorption, cells were washed with PBS, then incubated with DMEM containing 2% FBS and 10% fresh SPF chicken egg allantoic fluid at 37° C. in presence of 5% CO₂. Aliquots of 200 μL of supernatant from infected cells were collected and replaced with fresh DMEM including FBS at intervals of 8 hours until 56 hours post-infection. The titer of virus in the harvested samples was determined by TCID₅₀ method in DF-1 cells in 96-well tissue culture plates.

The protective efficacy of rNDVs expressing S1, S2 or S protein of IBV against virulent IBV challenge.

Based on the level of expression of S1, S2 and S proteins of IBV from rNDVs, rNDV/S1(cs+)+NDV-F-TM&CT, rNDV/S2, and rNDV/codon optimized-S viruses were selected for animal study to evaluate their protective efficacy against virulent IBV challenge.

IBV protection experiment 1.

In this study, the protective efficacy of rNDVs expressing S1, S2 or S protein of IBV strain Mass-41 were evaluated in 1-day-old specific pathogen free (SPF) chicks. Briefly, a total of eighty 1-day-old chicks were divided into five groups of fifteen each and one group of five. Chicks of the first four groups were inoculated with 10⁷ EID₅₀ of rNDV, rNDV/S1(cs+)+NDV-F-TM&CT, rNDV/S2 and rNDV/codon optimized-S strains via oculonasal route. The fifteen chicks of group five and five chicks of group six were inoculated with PBS. Three weeks after immunization, all immunized chickens, were challenged with 10^{3.1} EID₅₀ of virulent IBV strain Mass-41. This challenge virus dose was determined by an experimental chicken infection study. The severity scores of clinical signs of IBV including, nasal discharge, ocular discharge and difficulty in breathing (0=normal to 3=heavy ocular discharge and heavy nasal discharge with mouth breathing) were recorded twice a day for 10 days post-challenge. In order to evaluate protective efficacy of rNDVs expressing S1, S2 and S genes of IBV in preventing shedding of virulent IBV in immunized chickens, at day five post-challenge, tracheal swab samples were collected from fifteen birds of each group and placed in 2 mL serum free DMEM with 10× antibiotic. The swab samples were analyzed for quantification of viral RNA using an IBV-N gene-specific RT-qPCR.

IBV protection experiment 2.

In this study, the protective efficacy of rNDV expressing codon optimized S protein of IBV was evaluated in 4-week-old SPF chickens against the World Organization for Animal Health (OIE) recommended dose of virulent IBV challenge¹. A total of twenty 4-week-old SPF chickens were divided into four groups of five each. Five chickens of groups one and two were inoculated with 10⁷ EID₅₀ of rNDV and rNDV/codon optimized-S, respectively, via oculanasal route. Five chickens of group three were inoculated with recommended dose of a commercial live attenuated Mass-type IBV vaccine via oculanasal route and chickens of group four were inoculated with PBS. Three weeks after immunization, chickens of all groups were challenged with 10^{3.1} EID₅₀ of virulent IBV strain Mass-41 by the oculonasal route. The severity scores of clinical signs of IBV including, nasal discharge, ocular discharge and difficulty in breathing (0=normal to 3=heavy ocular discharge and heavy nasal discharge with mouth breathing) were recorded for 10 days post-challenge. In order to evaluate the efficacy of rNDV expressing S protein of IBV in preventing shedding of virulent IBV in immunized chickens, at day 5 post-challenge, tracheal swab samples were collected from twenty chickens and placed in 2 ml serum free DMEM with 10× antibiotic. The swab samples were analyzed for quantification of viral RNA using an IBV-N gene-specific RT-qPCR.

IBV protection experiment 3.

In this study, the protective efficacy of rNDV expressing codon optimized S protein of IBV was evaluated in 4-week-old SPF chickens against a higher dose of virulent IBV challenge. A total of twenty 4-week-old SPF chickens were divided into four groups of five each. Five chickens of group one and two were inoculated with 10⁷ EID₅₀ of rNDV and rNDV/codon optimized-S, respectively, via oculanasal route. Five chickens of group three were inoculated with recommended dose of a commercial live attenuated Mass-type IBV vaccine via oculanasal route and chickens of group four were inoculated with PBS. Three weeks after immunization, chickens of all groups were challenged with 10⁴⁷ EID₅₀ of virulent IBV strain Mass-41 by the oculonasal route. The severity scores of clinical signs of IBV including, nasal discharge, ocular discharge and difficulty in breathing (0=normal to 3=heavy ocular discharge and heavy nasal discharge with mouth breathing) were recorded for 10 days post-challenge. In order to evaluate the efficacy of rNDV expressing S protein of IBV in preventing shedding of virulent IBV in immunized chickens, at day 4 post-challenge, tracheal swab samples were collected from twenty chickens and placed in 2 mL serum free DMEM with 10× antibiotic. Each fluid was tested for IBV specific lesions on chicken embryo by inoculation (0.1 ml) of one 10-day-old embryonated SPF chicken egg.

The protective efficacy of rNDV expressing S protein of IBV against virulent NDV challenge.

The protective efficacy of rNDV expressing S protein of IBV strain Mass-41 was evaluated against a virulent NDV strain GB Texas challenge in our biosafety level 3 (BSL-3) plus facility. Briefly, a total of twenty 4-week-old chickens were divided into four groups of five each. Chickens of two groups were inoculated with 10⁷ EID₅₀ of rNDV and rNDV/IBV-codon optimized-S via oculonasal route. The five chickens of group three were inoculated with commercial IBV vaccine. The five chickens of group four were inoculated with PBS. Three weeks after immunization, blood samples of all birds were collected for NDV antibody response analysis and challenged with one hundred 50% chicken lethal dose (CLD₅₀) of the highly virulent NDV strain GB Texas via oculonasal route. The chickens were observed daily for 10 days after challenge for clinical signs of disease and mortality.

Serological analysis.

The level of antibodies induced against NDV and IBV were evaluated. The serum samples were collected three weeks post-immunization. Hemagglutination inhibition (HI) assay using a standard protocol OIE was used to assess the level of antibody titer mounted against NDV in chickens immunized by rNDVs′. The virus neutralization assay according to OIE was used to measure the level of neutralizing antibodies mounted against IBV′. Briefly, serum samples of three birds from the group immunized with rNDV expressing codon optimized S protein of IBV and serum samples of three birds from the group immunized with commercial IBV vaccine group were incubated at 56° C. for 30 minutes. One hundred EID₅₀ of IBV strain Mass-41 was mixed with 2 fold dilutions of antiserum and incubated for 1 hour at 37° C. One hundred μL of each serum and virus mixture was inoculated into three 10-day-old embryonated SPF chicken eggs. To confirm that at least 100 EID₅₀ of virus was inoculated into each egg, three eggs were inoculated with 100 μl of PBS containing 100 EID₅₀ of IBV. Three eggs were inoculated with 100 μL of PBS as negative control. Three eggs were inoculated with a mixture of 100 EID₅₀ of IBV and a dilution of 1:8 of a randomly selected serum sample collected from a bird immunized with rNDV strain LaSota as vector control. The eggs were incubated at 37° C. and were observed daily for dead chicken embryos for 7 days post inoculation. The serum titers were calculated according to the method of Reed and Muench³¹, based on mortality and IBV specific lesions on chicken embryos.

Quantitative reverse transcription-polymerase chain reaction (RT-qPCR).

RNA was extracted using TRIzol Reagent (Invitrogen) from tracheal swab samples collected from chickens. The first strand cDNA was synthesized using Thermo Scientific RevertAid Reverse Transcriptase (RT). SYBR green RT-qPCR was performed using a specific primer pair set: a) N gene—296 forward primer: 5′ GACCAGCCGCTAACCTGAAT 3′ (SEQ ID NO:5) and b) N gene—445 reverse primer: 5′ GTCCTCCGTCTGAAAACCGT 3′ (SEQ ID NO:6) amplifying 150 nt of N gene of IBV strain Mass-41. PCRs were performed using a Bio-Rad CFX96 Cycler. Each 20 μl reaction was carried out using 5 μl of cDNA, 10 μl of iTaq Universal SYBR Green Supermix (Bio-Rad), 2 μl of forward and reverse primers and 3 μl of nuclease free water. Forty cycles of PCR at 95° C. for 10 s (denaturation), 55° C. for 20 s (annealing), and 72° C. for 30 s (elongation) followed by melting curve analysis that consisted of 95° C. for 5 s and 65° C. for 60 s. A serial 10 fold dilution of cDNA synthesized from extracted RNA of allantoic fluid stock of a virulent IBV strain Mass-41 with 10⁷⁵ EID₅₀/ml was used to establish the standard curve. The cDNA synthesized from extracted RNA of allantoic fluid stock of a virulent IBV strain Mass-41 and the cDNA synthesized from extracted RNA of swab sample solution were served as positive and negative controls, respectively. Melting point analysis was used to confirm the specificity of the test.

Statistical analysis.

Data were analyzed among groups by One-Way-ANOVA test. The student t-test was used to compare two groups. To avoid bias, all animal experiments were designed as blinded studies.

The following sequences are provided and are used to represent distinct but not limiting embodiments of this disclosure.

In the figures and text of this disclosure the following abbreviations are used: “SP” is the “signal peptide” of S protein of IBV which is involved in secretion of the protein. “GE” (gene end) and “GS” (gene start) are approximately ten conserved sequences, which are transcriptional signals of NDV used to express any foreign gene by NDV polymerase. These sequences are not translated. Before the initiating Met in certain vectors and protein sequences described herein, the expression cassette includes: a PmeI restriction enzyme site sequence (GTTTAAAC), 15 nt of UTR of NDV P gene (tagctacatttaaga SEQ ID NO:7; AF077761.1), gene end (GE) signal of NDV P gene (TTAAGAAAAAA SEQ ID NO:8; AF077761.1), one “t” nucleotide as intergenic sequence (IG), gene start(GS) signal of NDV M gene (ACGGGTAGAA SEQ ID NO:9; AF077761.1), nucleotides for maintaining rule of six (preferable for some expression cassettes), and a Kozak sequence (gccacc).

“TM” is the transmembrane and “CT” is the cytoplasmic tail of F protein of NDV or S protein of IBV, which is exchanged with the corresponding sequences of the foreign protein for incorporation into NDV envelope. “Foreign ORF” is the open reading frame of any foreign gene, which in the embodiments demonstrated herein are the S1, S2 and S gene. The DNA sequences presented below are cDNA sequences of rNDV genome and IBV genes RNA sequences. The “insertion site” is the site in the NDV genome in which the foreign gene (S1, S2 or S as demonstrated herein). In the examples provided herein the insertion site is between the NVD P and M genes.

S protein of IBV strain Mass-41 (Accession
number: AY851295.1)
SEQ ID NO: 10
MLVTPLLLVTLLCVLCSAALYDSSSYVYYYQSAFRPPNGWHLHGGAYAVV
NISSESNNAGSSPGCIVGTIHGGRVVNASSIAMTAPSSGMAWSSSQFCTA
HCNFSDTTVFVTHCYKYDGCPITGMLQKNFLRYSAMKNGQLFYNLTVSVA
KYPTFKSFQCVNNLTSVYLNGDLVYTSNETTDVTSAGVYFKAGGPITYKV
MREVKALAYFVNGTAQDVILCDGSPRGLLACQYNTGNFSDGFYPFINSSL
VKQKFIVYRENSVNTTFTLHNFTFHNETGANPNPSGVQNIQTYQTQTAQS
GYYNFNFSFLSSFVYKESNFMYGSYHPSCNFRLETINNGLWFNSLSVSIA
YGPLQGGCKQSVFSGRATCCYAYSYGGPSLCKGVYSGELDLNFECGLLVY
VTKSGGSRIQTATEPPVITRHNYNNITLNTCVDYNIYGRTGQGFITNVTD
SAVSYNYLADAGLAILDTSGSIDIFVVQGEYGLTYYKVNPCEDVNQQFVV
SGGKLVGILTSRNETGSQLLENQFYIKITNG [symbol]
SITENVANCPYVSYGKFCIKPDGSIATIVPKQLEQFVAPLLNVTENVLIP
NSFNLTVTDEYIQTRMDKVQINCLQYVCGNSLDCRDLFQQYGPVCDNILS
VVNSIGQKEDMELLNFYSSTKPAGFNTPFLSNVSTGEFNISLLLTTPSSP
RRRSFIEDLLFTSVESVGLPTDDAYKNCTAGPLGFLKDLACAREYNGLLV
LPPIITAEMQTLYTSSLVASMAFGGITAAGAIPFATQLQARINHLGITQS
LLLKNQEKIAASENKAIGRMQEGFRSTSLALQQIQDVVNKQSAILTETMA
SLNKNFGAISSVIQEIYQQLDAIQANAQVDRLITGRLSSLSVLASAKQAE
HIRVSQQRELATQKINECVKSQSIRYSFCGNGRHVLTIPQNAPNGIVFIH
FSYTPDSFVNVTAIVGFCVKPANASQYAIVPANGRGIFIQVNGSYYITAR
DMYMPRAITAGDIVTLTSCQANYVSVNKTVITTFVDNDDFDENDELSKWW
NDTKHELPDFDKFNYTVPILDIDSEIDRIQGVIQGLNDSLIDLEKLSILK
TYIKWPWYVWLAIAFATIIFILILGWVFFMTGC
CGCCCGCFGIMPLMSKCGKKSSYYTTFDNDVVTEQNRPKKSV.
Bold: S1 amino acid sequences (SEQ ID NO: 11)
Italics: S2 amino acid sequences (SEQ ID NO: 13)
Plain and enlarged: cleavage site sequence
(SEQ ID NO: 12)

In the following DNA sequences the italicized sequences are the transmembrane (TM) and cytoplasmic tail (CT) sequences. Lower case letters indicate insertion sites. Each sequence is flanked at its ends by a PmeI restriction site. Where indicated in lower case letters the UTR of NDV P gene (tagctacatttaaga) (SEQ ID NO:7) is shown. Also shown in lower case where indicated are Kozak sequence (gccacc). Initiation codons are enlarged.

IBV-Mass-41 codon optimized S cassette (P-M insertion site)
SEQ ID NO: 14
GTTTAAACtagctacatttaagaTTAAGAAAAAAtACGGGTAGAAgccaccATGCTGGTGACTCCACTGCTGCTGGTGACTC
TGCTGTGCGTGCTGTGTTCCGCCGCCCTGTACGATTCATCATCATACGTGTACTATTACCAGTCCGCTTTTAGGCCCCCTAA
TGGATGGCACCTGCACGGCGGAGCTTACGCAGTGGTGAATATCAGCTCCGAGAGCAACAATGCTGGAAGCTCCCCTGGATGC
ATTGTGGGAACTATTCACGGCGGCAGAGTGGTGAACGCTAGCTCCATTGCCATGACCGCTCCAAGCTCCGGAATGGCCTGGA
GCTCCAGCCAGTTCTGCACAGCTCACTGCAATTTTAGCGATACCACAGTGTTCGTGACCCACTGCTACAAGTACGACGGCTG
CCCAATCACAGGAATGCTGCAGAAGAACTTTCTGAGGGTGAGCGCCATGAAAAACGGCCAGCTGTTCTACAATCTGACTGTG
AGCGTGGCTAAGTACCCCACCTTTAAATCCTTCCAGTGCGTGAACAATCTGACCAGCGTGTACCTGAACGGCGATCTGGTGT
ACACAAGCAATGAGACTACCGACGTGACTTCCGCCGGAGTGTACTTTAAAGCTGGAGGGCCTATCACATACAAGGTGATGAG
AGAAGTGAAAGCCCTGGCTTACTTCGTGAACGGCACTGCACAGGATGTGATTCTGTGCGATGGCTCCCCACGCGGACTGCTG
GCCTGCCAGTACAACACCGGGAATTTCAGCGATGGCTTTTACCCCTTCATCAACTCCAGCCTGGTGAAGCAGAAATTTATTG
TGTACAGAGAGAACTCCGTGAATACAACTTTCACACTGCACAACTTCACTTTTCACAATGAAACCGGCGCTAACCCCAATCC
TAGCGGAGTGCAGAACATCCAGACATACCAGACACAGACTGCACAGAGCGGGTACTACAACTTCAATTTTTCCTTCCTGTCC
AGCTTTGTGTACAAGGAGAGCAACTTCATGTACGGCAGCTACCACCCATCCTGCAATTTTCGGCTGGAAACAATCAACAATG
GACTGTGGTTCAACTCCCTGAGCGTGTCCATTGCATACGGGCCCCTGCAGGGCGGATGCAAACAGAGCGTGTTTTCCGGCAG
AGCTACTTGCTGCTACGCATACAGCTACGGCGGACCTTCCCTGTGCAAGGGGGTGTACAGCGGCGAGCTGGATCTGAATTTC
GAATGCGGACTGCTGGTGTACGTGACCAAAAGCGGAGGGTCCAGGATCCAGACCGCCACAGAGCCACCCGTGATCACACGCC
ACAACTACAACAATATTACTCTGAACACCTGCGTGGACTACAATATCTACGGGAGGACAGGACAGGGGTTCATTACAAACGT
GACTGATAGCGCAGTGTCCTACAATTACCTGGCAGATGCTGGGCTGGCAATCCTGGATACTAGCGGCTCCATCGACATTTTT
GTGGTGCAGGGAGAGTACGGGCTGACCTACTACAAGGTGAACCCCTGCGAAGATGTGAATCAGCAGTTCGTGGTGAGCGGCG
GAAAACTGGTGGGCATCCTGACAAGCCGCAACGAGACTGGATCCCAGCTGCTGGAAAACCAGTTTTACATCAAGATTACCAA
TGGAACAAGAAGGTTCAGAAGAAGCATCACCGAGAACGTGGCTAATTGCCCCTACGTGTCCTACGGAAAGTTTTGCATTAAA
CCAGACGGGAGCATCGCTACTATTGTGCCCAAGCAGCTGGAGCAGTTTGTGGCACCTCTGCTGAACGTGACCGAAAATGTGC
TGATCCCTAACAGCTTCAATCTGACTGTGACCGATGAATACATCCAGACACGGATGGACAAAGTGCAGATTAACTGCCTGCA
GTACGTGTGCGGAAATAGCCTGGATTGCAGAGATCTGTTCCAGCAGTACGGGCCTGTGTGCGATAACATCCTGAGCGTGGTG
AATTCCATTGGCCAGAAAGAGGATATGGAACTGCTGAACTTTTACTCCAGCACCAAACCTGCCGGCTTTAACACACCTTTCC
TGAGCAATGTGTCCACCGGCGAGTTCAATATCTCCCTGCTGCTGACCACACCATCCAGCCCCAGAAGGCGCAGCTTTATTGA
GGATCTGCTGTTCACAAGCGTGGAATCCGTGGGGCTGCCCACCGATGACGCATACAAGAACTGCACAGCCGGCCCTCTGGGA
TTCCTGAAAGACCTGGCTTGCGCACGGGAGTACAATGGCCTGCTGGTGCTGCCTCCAATCATTACCGCCGAAATGCAGACTC
TGTACACCTCCAGCCTGGTGGCAAGCATGGCCTTTGGCGGAATCACAGCCGCTGGGGCCATTCCCTTCGCTACTCAGCTGCA
GGCTAGAATCAACCACCTGGGCATTACTCAGTCCCTGCTGCTGAAGAACCAGGAGAAAATCGCAGCCAGCTTTAATAAGGCA
ATTGGACGGATGCAGGAAGGGTTCAGAAGCACCTCCCTGGCCCTGCAGCAGATCCAGGATGTGGTGAACAAGCAGTCCGCTA
TTCTGACAGAGACTATGGCAAGCCTGAACAAAAATTTTGGCGCTATCTCCAGCGTGATCCAGGAAATTTACCAGCAGCTGGA
TGCAATCCAGGCCAACGCTCAGGTGGACCGGCTGATTACAGGAAGACTGTCCAGCCTGAGCGTGCTGGCATCCGCAAAGCAG
GCTGAGCACATCAGGGTGTCCCAGCAGCGCGAGCTGGCTACACAGAAGATCAACGAATGCGTGAAAAGCCAGTCCATTCGGT
ACAGCTTCTGCGGCAATGGAAGACACGTGCTGACTATCCCTCAGAACGCACCAAATGGCATCGTGTTTATTCACTTCAGCTA
CACTCCAGACTCCTTTGTGAACGTGACCGCCATCGTGGGATTCTGCGTGAAGCCAGCTAATGCATCCCAGTACGCAATTGTG
CCCGCCAACGGCAGAGGCATCTTTATTCAAGTGAATGGAAGCTACTACATCACTGCTAGGGATATGTACATGCCCAGAGCTA
TCACCGCAGGGGACATTGTGACCCTGACAAGCTGCCAGGCAAACTACGTGTCCGTGAATAAAACCGTGATTACTACCTTCGT
GGATAACGATGACTTTGATTTCAATGACGAGCTGTCCAAGTGGTGGAACGACACAAAACACGAACTGCCTGATTTTGACAAG
TTCAATTACACTGTGCCAATCCTGGATATTGACAGCGAGATCGACCGCATTCAGGGAGTGATCCAGGGGCTGAACGATAGCC
TGATTGACCTGGAAAAACTGTCCATCCTGAAGACATACATTAAATGGCCTTGGTACGTGTGGCTGGCCATCGCTTTTGCAAC
CATCATTTTCATCCTGATTCTGGGATGGGTGTTCTTTATGACAGGGTGCTGCGGCTGCTGCTGCGGATGCTTCGGGATTATG
CCACTGATGAGCAAGTGCGGGAAGAAATCCAGCTACTATACAACCTTTGACAATGATGTGGTGACAGAGCAGAATCGCCCTA
AGAAATCCGTGTGAGTTTAAAC
IBV-Mass-41 codon optimized S1 cassette (P-M insertion site)
SEQ ID NO: 15
GTTTAAACTTAAGAAAAAAtACGGGTAGAAgccacc [symbol] CTGGTGACTCCACTGCTGCT
GGTGACTCTGCTGTGCGTGCTGTGTTCCGCCGCCCTGTACGATTCATCATCATACGTGTACTATTACCAGTCCGCTTTTAGG
CCCCCTAATGGATGGCACCTGCACGGCGGAGCTTACGCAGTGGTGAATATCAGCTCCGAGAGCAACAATGCTGGAAGCTCCC
CTGGATGCATTGTGGGAACTATTCACGGCGGCAGAGTGGTGAACGCTAGCTCCATTGCCATGACCGCTCCAAGCTCCGGAAT
GGCCTGGAGCTCCAGCCAGTTCTGCACAGCTCACTGCAATTTTAGCGATACCACAGTGTTCGTGACCCACTGCTACAAGTAC
GACGGCTGCCCAATCACAGGAATGCTGCAGAAGAACTTTCTGAGGGTGAGCGCCATGAAAAACGGCCAGCTGTTCTACAATC
TGACTGTGAGCGTGGCTAAGTACCCCACCTTTAAATCCTTCCAGTGCGTGAACAATCTGACCAGCGTGTACCTGAACGGCGA
TCTGGTGTACACAAGCAATGAGACTACCGACGTGACTTCCGCCGGAGTGTACTTTAAAGCTGGAGGGCCTATCACATACAAG
GTGATGAGAGAAGTGAAAGCCCTGGCTTACTTCGTGAACGGCACTGCACAGGATGTGATTCTGTGCGATGGCTCCCCACGCG
GACTGCTGGCCTGCCAGTACAACACCGGGAATTTCAGCGATGGCTTTTACCCCTTCATCAACTCCAGCCTGGTGAAGCAGAA
ATTTATTGTGTACAGAGAGAACTCCGTGAATACAACTTTCACACTGCACAACTTCACTTTTCACAATGAAACCGGCGCTAAC
CCCAATCCTAGCGGAGTGCAGAACATCCAGACATACCAGACACAGACTGCACAGAGCGGGTACTACAACTTCAATTTTTCCT
TCCTGTCCAGCTTTGTGTACAAGGAGAGCAACTTCATGTACGGCAGCTACCACCCATCCTGCAATTTTCGGCTGGAAACAAT
CAACAATGGACTGTGGTTCAACTCCCTGAGCGTGTCCATTGCATACGGGCCCCTGCAGGGCGGATGCAAACAGAGCGTGTTT
TCCGGCAGAGCTACTTGCTGCTACGCATACAGCTACGGCGGACCTTCCCTGTGCAAGGGGGTGTACAGCGGCGAGCTGGATC
TGAATTTCGAATGCGGACTGCTGGTGTACGTGACCAAAAGCGGAGGGTCCAGGATCCAGACCGCCACAGAGCCACCCGTGAT
CACACGCCACAACTACAACAATATTACTCTGAACACCTGCGTGGACTACAATATCTACGGGAGGACAGGACAGGGGTTCATT
ACAAACGTGACTGATAGCGCAGTGTCCTACAATTACCTGGCAGATGCTGGGCTGGCAATCCTGGATACTAGCGGCTCCATCG
ACATTTTTGTGGTGCAGGGAGAGTACGGGCTGACCTACTACAAGGTGAACCCCTGCGAAGATGTGAATCAGCAGTTCGTGGT
GAGCGGCGGAAAACTGGTGGGCATCCTGACAAGCCGCAACGAGACTGGATCCCAGCTGCTGGAAAACCAGTTTTACATCAAG
ATTACCAATGGAACAAGAAGGTTCAGAAGATGAGTTTAAAC
IBV-Mass-41 codon optimized S1 + IBV TM & CT cassette (P-M insertion site)
SEQ ID NO: 16
GTTTAAACtagctacatttaagaTTAAGAAAAAAtACGGGTAGAAgccacc [symbol] CTG
GTGACTCCACTGCTGCTGGTGACTCTGCTGTGCGTGCTGTGTTCCGCCGCCCTGTACGATTCATCATCATACGTGTACTATT
ACCAGTCCGCTTTTAGGCCCCCTAATGGATGGCACCTGCACGGCGGAGCTTACGCAGTGGTGAATATCAGCTCCGAGAGCAA
CAATGCTGGAAGCTCCCCTGGATGCATTGTGGGAACTATTCACGGCGGCAGAGTGGTGAACGCTAGCTCCATTGCCATGACC
GCTCCAAGCTCCGGAATGGCCTGGAGCTCCAGCCAGTTCTGCACAGCTCACTGCAATTTTAGCGATACCACAGTGTTCGTGA
CCCACTGCTACAAGTACGACGGCTGCCCAATCACAGGAATGCTGCAGAAGAACTTTCTGAGGGTGAGCGCCATGAAAAACGG
CCAGCTGTTCTACAATCTGACTGTGAGCGTGGCTAAGTACCCCACCTTTAAATCCTTCCAGTGCGTGAACAATCTGACCAGC
GTGTACCTGAACGGCGATCTGGTGTACACAAGCAATGAGACTACCGACGTGACTTCCGCCGGAGTGTACTTTAAAGCTGGAG
GGCCTATCACATACAAGGTGATGAGAGAAGTGAAAGCCCTGGCTTACTTCGTGAACGGCACTGCACAGGATGTGATTCTGTG
CGATGGCTCCCCACGCGGACTGCTGGCCTGCCAGTACAACACCGGGAATTTCAGCGATGGCTTTTACCCCTTCATCAACTCC
AGCCTGGTGAAGCAGAAATTTATTGTGTACAGAGAGAACTCCGTGAATACAACTTTCACACTGCACAACTTCACTTTTCACA
ATGAAACCGGCGCTAACCCCAATCCTAGCGGAGTGCAGAACATCCAGACATACCAGACACAGACTGCACAGAGCGGGTACTA
CAACTTCAATTTTTCCTTCCTGTCCAGCTTTGTGTACAAGGAGAGCAACTTCATGTACGGCAGCTACCACCCATCCTGCAAT
TTTCGGCTGGAAACAATCAACAATGGACTGTGGTTCAACTCCCTGAGCGTGTCCATTGCATACGGGCCCCTGCAGGGCGGAT
GCAAACAGAGCGTGTTTTCCGGCAGAGCTACTTGCTGCTACGCATACAGCTACGGCGGACCTTCCCTGTGCAAGGGGGTGTA
CAGCGGCGAGCTGGATCTGAATTTCGAATGCGGACTGCTGGTGTACGTGACCAAAAGCGGAGGGTCCAGGATCCAGACCGCC
ACAGAGCCACCCGTGATCACACGCCACAACTACAACAATATTACTCTGAACACCTGCGTGGACTACAATATCTACGGGAGGA
CAGGACAGGGGTTCATTACAAACGTGACTGATAGCGCAGTGTCCTACAATTACCTGGCAGATGCTGGGCTGGCAATCCTGGA
TACTAGCGGCTCCATCGACATTTTTGTGGTGCAGGGAGAGTACGGGCTGACCTACTACAAGGTGAACCCCTGCGAAGATGTG
AATCAGCAGTTCGTGGTGAGCGGCGGAAAACTGGTGGGCATCCTGACAAGCCGCAACGAGACTGGATCCCAGCTGCTGGAAA
ACCAGTTTTACATCAAGATTACCAATGGAACAAGAAGGTTCAGAAGAATTGACCTGGAAAAACTGTCCATCCTGAAGACATA
CATTAAATGGCCTTGGTACGTGTGGCTGGCCATCGCTTTTGCAACCATCATTTTCATCCTGATTCTGGGATGGGTGTTCTTT
ATGACAGGGTGCTGCGGCTGCTGCTGCGGATGCTTCGGGATTATGCCACTGATGAGCAAGTGCGGGAAGAAATCCAGCTACT
ATACAACCTTTGACAATGATGTGGTGACAGAGCAGAATCGCCCTAAGAAATCCGTGTGAGTTTAAAC
IBV-Mass-41 codon optimized S1 (containing S cleavage site) + NDV F TM & CT
cassette (P-M insertion site)
SEQ ID NO: 17
GTTTAAACtagctacatttaagaTTAAGAAAAAAtACGGGTAGAAgccacc [symbol] C
TGGTGACTCCACTGCTGCTGGTGACTCTGCTGTGCGTGCTGTGTTCCGCCGCCCTGTACGATTCATCATCATACGTGTACTA
TTACCAGTCCGCTTTTAGGCCCCCTAATGGATGGCACCTGCACGGCGGAGCTTACGCAGTGGTGAATATCAGCTCCGAGAGC
AACAATGCTGGAAGCTCCCCTGGATGCATTGTGGGAACTATTCACGGCGGCAGAGTGGTGAACGCTAGCTCCATTGCCATGA
CCGCTCCAAGCTCCGGAATGGCCTGGAGCTCCAGCCAGTTCTGCACAGCTCACTGCAATTTTAGCGATACCACAGTGTTCGT
GACCCACTGCTACAAGTACGACGGCTGCCCAATCACAGGAATGCTGCAGAAGAACTTTCTGAGGGTGAGCGCCATGAAAAAC
GGCCAGCTGTTCTACAATCTGACTGTGAGCGTGGCTAAGTACCCCACCTTTAAATCCTTCCAGTGCGTGAACAATCTGACCA
GCGTGTACCTGAACGGCGATCTGGTGTACACAAGCAATGAGACTACCGACGTGACTTCCGCCGGAGTGTACTTTAAAGCTGG
AGGGCCTATCACATACAAGGTGATGAGAGAAGTGAAAGCCCTGGCTTACTTCGTGAACGGCACTGCACAGGATGTGATTCTG
TGCGATGGCTCCCCACGCGGACTGCTGGCCTGCCAGTACAACACCGGGAATTTCAGCGATGGCTTTTACCCCTTCATCAACT
CCAGCCTGGTGAAGCAGAAATTTATTGTGTACAGAGAGAACTCCGTGAATACAACTTTCACACTGCACAACTTCACTTTTCA
CAATGAAACCGGCGCTAACCCCAATCCTAGCGGAGTGCAGAACATCCAGACATACCAGACACAGACTGCACAGAGCGGGTAC
TACAACTTCAATTTTTCCTTCCTGTCCAGCTTTGTGTACAAGGAGAGCAACTTCATGTACGGCAGCTACCACCCATCCTGCA
ATTTTCGGCTGGAAACAATCAACAATGGACTGTGGTTCAACTCCCTGAGCGTGTCCATTGCATACGGGCCCCTGCAGGGCGG
ATGCAAACAGAGCGTGTTTTCCGGCAGAGCTACTTGCTGCTACGCATACAGCTACGGCGGACCTTCCCTGTGCAAGGGGGTG
TACAGCGGCGAGCTGGATCTGAATTTCGAATGCGGACTGCTGGTGTACGTGACCAAAAGCGGAGGGTCCAGGATCCAGACCG
CCACAGAGCCACCCGTGATCACACGCCACAACTACAACAATATTACTCTGAACACCTGCGTGGACTACAATATCTACGGGAG
GACAGGACAGGGGTTCATTACAAACGTGACTGATAGCGCAGTGTCCTACAATTACCTGGCAGATGCTGGGCTGGCAATCCTG
GATACTAGCGGCTCCATCGACATTTTTGTGGTGCAGGGAGAGTACGGGCTGACCTACTACAAGGTGAACCCCTGCGAAGATG
TGAATCAGCAGTTCGTGGTGAGCGGCGGAAAACTGGTGGGCATCCTGACAAGCCGCAACGAGACTGGATCCCAGCTGCTGGA
AAACCAGTTTTACATCAAGATTACCAATGGAACAAGAAGGTTCAGAAGAagcacatctgctctcattacctatatcgttttg
actatcatatctcttgtttttggtatacttagcctgattctagcatgctacctaatgtacaagcaaaaggcgcaacaaaaga
ccttattatggcttgggaataatactctagatcagatgagagccactacaaaaatgtgaGTTTAAAC
IBV-Mass-41 codon optimized S1 (without S cleavage site) + NDV F TM & CT cassette
(P-M insertion site)
SEQ ID NO: 18
GTTTAAACtagctacatttaagaTTAAGAAAAAAtACGGGTAGAAgccacc [symbol] CTG
GTGACTCCACTGCTGCTGGTGACTCTGCTGTGCGTGCTGTGTTCCGCCGCCCTGTACGATTCATCATCATACGTGTACTATT
ACCAGTCCGCTTTTAGGCCCCCTAATGGATGGCACCTGCACGGCGGAGCTTACGCAGTGGTGAATATCAGCTCCGAGAGCAA
CAATGCTGGAAGCTCCCCTGGATGCATTGTGGGAACTATTCACGGCGGCAGAGTGGTGAACGCTAGCTCCATTGCCATGACC
GCTCCAAGCTCCGGAATGGCCTGGAGCTCCAGCCAGTTCTGCACAGCTCACTGCAATTTTAGCGATACCACAGTGTTCGTGA
CCCACTGCTACAAGTACGACGGCTGCCCAATCACAGGAATGCTGCAGAAGAACTTTCTGAGGGTGAGCGCCATGAAAAACGG
CCAGCTGTTCTACAATCTGACTGTGAGCGTGGCTAAGTACCCCACCTTTAAATCCTTCCAGTGCGTGAACAATCTGACCAGC
GTGTACCTGAACGGCGATCTGGTGTACACAAGCAATGAGACTACCGACGTGACTTCCGCCGGAGTGTACTTTAAAGCTGGAG
GGCCTATCACATACAAGGTGATGAGAGAAGTGAAAGCCCTGGCTTACTTCGTGAACGGCACTGCACAGGATGTGATTCTGTG
CGATGGCTCCCCACGCGGACTGCTGGCCTGCCAGTACAACACCGGGAATTTCAGCGATGGCTTTTACCCCTTCATCAACTCC
AGCCTGGTGAAGCAGAAATTTATTGTGTACAGAGAGAACTCCGTGAATACAACTTTCACACTGCACAACTTCACTTTTCACA
ATGAAACCGGCGCTAACCCCAATCCTAGCGGAGTGCAGAACATCCAGACATACCAGACACAGACTGCACAGAGCGGGTACTA
CAACTTCAATTTTTCCTTCCTGTCCAGCTTTGTGTACAAGGAGAGCAACTTCATGTACGGCAGCTACCACCCATCCTGCAAT
TTTCGGCTGGAAACAATCAACAATGGACTGTGGTTCAACTCCCTGAGCGTGTCCATTGCATACGGGCCCCTGCAGGGCGGAT
GCAAACAGAGCGTGTTTTCCGGCAGAGCTACTTGCTGCTACGCATACAGCTACGGCGGACCTTCCCTGTGCAAGGGGGTGTA
CAGCGGCGAGCTGGATCTGAATTTCGAATGCGGACTGCTGGTGTACGTGACCAAAAGCGGAGGGTCCAGGATCCAGACCGCC
ACAGAGCCACCCGTGATCACACGCCACAACTACAACAATATTACTCTGAACACCTGCGTGGACTACAATATCTACGGGAGGA
CAGGACAGGGGTTCATTACAAACGTGACTGATAGCGCAGTGTCCTACAATTACCTGGCAGATGCTGGGCTGGCAATCCTGGA
TACTAGCGGCTCCATCGACATTTTTGTGGTGCAGGGAGAGTACGGGCTGACCTACTACAAGGTGAACCCCTGCGAAGATGTG
AATCAGCAGTTCGTGGTGAGCGGCGGAAAACTGGTGGGCATCCTGACAAGCCGCAACGAGACTGGATCCCAGCTGCTGGAAA
ACCAGTTTTACATCAAGATTACCAATGGAagcacatctgctctcattacctatatcgttttgactatcatatctcttgtttt
tggtatacttagcctgattctagcatgctacctaatgtacaagcaaaaggcgcaacaaaagaccttattatggcttgggaat
aatactctagatcagatgagagccactacaaaaatgtgaGTTTAAAC
IBV-Mass-41 codon optimized signal peptide (SP) + S2 cassette (P-M insertion
site) In this construct the SP (signal peptide of S protein has been fused
with S2 gene. AGC is the first codon of the S2 coding region.
SEQ ID NO: 19
GTTTAAACtagctacatttaagaTTAAGAAAAAAtACGGGTAGAAgccaccATGCTGGTGACTCCACTGCTGCTGGTGACTC
TGCTGTGCGTGCTGTGTTCCGCCGCCCTGTACGATTCA [symbol] ATCACCGAGAACGTGG
CTAATTGCCCCTACGTGTCCTACGGAAAGTTTTGCATTAAACCAGACGGGAGCATCGCTACTATTGTGCCCAAGCAGCTGGA
GCAGTTTGTGGCACCTCTGCTGAACGTGACCGAAAATGTGCTGATCCCTAACAGCTTCAATCTGACTGTGACCGATGAATAC
ATCCAGACACGGATGGACAAAGTGCAGATTAACTGCCTGCAGTACGTGTGCGGAAATAGCCTGGATTGCAGAGATCTGTTCC
AGCAGTACGGGCCTGTGTGCGATAACATCCTGAGCGTGGTGAATTCCATTGGCCAGAAAGAGGATATGGAACTGCTGAACTT
TTACTCCAGCACCAAACCTGCCGGCTTTAACACACCTTTCCTGAGCAATGTGTCCACCGGCGAGTTCAATATCTCCCTGCTG
CTGACCACACCATCCAGCCCCAGAAGGCGCAGCTTTATTGAGGATCTGCTGTTCACAAGCGTGGAATCCGTGGGGCTGCCCA
CCGATGACGCATACAAGAACTGCACAGCCGGCCCTCTGGGATTCCTGAAAGACCTGGCTTGCGCACGGGAGTACAATGGCCT
GCTGGTGCTGCCTCCAATCATTACCGCCGAAATGCAGACTCTGTACACCTCCAGCCTGGTGGCAAGCATGGCCTTTGGCGGA
ATCACAGCCGCTGGGGCCATTCCCTTCGCTACTCAGCTGCAGGCTAGAATCAACCACCTGGGCATTACTCAGTCCCTGCTGC
TGAAGAACCAGGAGAAAATCGCAGCCAGCTTTAATAAGGCAATTGGACGGATGCAGGAAGGGTTCAGAAGCACCTCCCTGGC
CCTGCAGCAGATCCAGGATGTGGTGAACAAGCAGTCCGCTATTCTGACAGAGACTATGGCAAGCCTGAACAAAAATTTTGGC
GCTATCTCCAGCGTGATCCAGGAAATTTACCAGCAGCTGGATGCAATCCAGGCCAACGCTCAGGTGGACCGGCTGATTACAG
GAAGACTGTCCAGCCTGAGCGTGCTGGCATCCGCAAAGCAGGCTGAGCACATCAGGGTGTCCCAGCAGCGCGAGCTGGCTAC
ACAGAAGATCAACGAATGCGTGAAAAGCCAGTCCATTCGGTACAGCTTCTGCGGCAATGGAAGACACGTGCTGACTATCCCT
CAGAACGCACCAAATGGCATCGTGTTTATTCACTTCAGCTACACTCCAGACTCCTTTGTGAACGTGACCGCCATCGTGGGAT
TCTGCGTGAAGCCAGCTAATGCATCCCAGTACGCAATTGTGCCCGCCAACGGCAGAGGCATCTTTATTCAAGTGAATGGAAG
CTACTACATCACTGCTAGGGATATGTACATGCCCAGAGCTATCACCGCAGGGGACATTGTGACCCTGACAAGCTGCCAGGCA
AACTACGTGTCCGTGAATAAAACCGTGATTACTACCTTCGTGGATAACGATGACTTTGATTTCAATGACGAGCTGTCCAAGT
GGTGGAACGACACAAAACACGAACTGCCTGATTTTGACAAGTTCAATTACACTGTGCCAATCCTGGATATTGACAGCGAGAT
CGACCGCATTCAGGGAGTGATCCAGGGGCTGAACGATAGCCTGATTGACCTGGAAAAACTGTCCATCCTGAAGACATACATT
AAATGGCCTTGGTACGTGTGGCTGGCCATCGCTTTTGCAACCATCATTTTCATCCTGATTCTGGGATGGGTGTTCTTTATGA
CAGGGTGCTGCGGCTGCTGCTGCGGATGCTTCGGGATTATGCCACTGATGAGCAAGTGCGGGAAGAAATCCAGCTACTATAC
AACCTTTGACAATGATGTGGTGACAGAGCAGAATCGCCCTAAGAAATCCGTGTGAGTTTAAAC
Fusion (F) protein of NDV strain LaSota (Accession number: AF077761.1)
SEQ ID NO: 20
MGSRPSTKNPAPMMLTIRVALVLSCICPANSIDGRPLAAAGIVVTGDKAVNIYTSSQTGSIIVKLLPNLPKDKEACAKAPLD
AYNRTLTTLLTPLGDSIRRIQESVTTSGGGRQGRLIGAIIGGVALGVATAAQITAAAALIQAKQNAANILRLKESIAATNEA
VHEVTDGLSQLAVAVGKMQQFVNDQFNKTAQELDCIKIAQQVGVELNLYLTELTTVFGPQITSPALNKLTIQALYNLAGGNM
DYLLTKLGVGNNQLSSLIGSGLITGNPILYDSQTQLLGIQVTLPSVGNLNNMRATYLETLSVSTTRGFASALVPKVVTQVGS
VIEELDTSYCIETDLDLYCTRIVTFPMSPGIYSCLSGNTSACMYSKTEGALTTPYMTIKGSVIANCKMTTCRCVNPPGIISQ
NYGEAVSLIDKQSCNVLSLGGITLRLSGEFDVTYQKNISIQDSQVIITGNLDISTELGNVNNSISNALNKLEESNRKLDKVN
VKLTSTSALITYIVLTIISLVFGILSLILACYLMYKQKAQQKTLLWLGNNTLDQMRATTKM

### REFERENCES

1. OIE. 2013. Chapter 2.3.2. Avian Infectious Bronchitis. Version adopted by the World Assembly of Delegates of the OIE in May 2013. In OIE terrestrial manual 2013.www.oie.int/fileadmin/Home/eng/Health_standards/tahm/2.03.02_AIB.pdf
2. Cook, J. K. A., Jackwood M. & Jones R. C. The long view: 40 years of infectious bronchitis research. Avian Pathology. 41, 239-250 (2014).
3. Cavanagh, D. & Gelb, J. Jr. Infectious bronchitis. In Diseases of Poultry (ed. Saif, Y. M et al.) 117-135 (Ames, Iowa: Blackwell Publishing, 2008).
4. Cavanagh D, Coronavirus avian infectious bronchitis virus. Veterinary Research. 38, 281-297 (2007).
5. Belouzard, S., Millet, J. K., Licitra, B. N. & Whittaker, G. R. Mechanisms of coronavirus cell entry mediated by the viral spike protein. Viruses. 4, 1011-1033(2012).
6. Ignjatovic, J. & Sapats, S. Identification of previously unknown antigenic epitopes on the S and N proteins of avian infectious bronchitis virus. Arch Virol. 150, 1813-1831(2005).
7. Niesters H. G. et al. The neutralization epitopes on the spike protein of infectious bronchitis virus and their antig enic variation. Adv Exp Med Biol. 218, 483-492(1987).
8. Promkuntod N., Van Eijndhoven R. E., De Vrieze G., Grone A. & Verheije M. H. Mapping of the receptor-binding domain and amino acids critical for attachment in the spike protein of avian coronavirus infectious bronchitis virus. Virology. 448:26-32(2014).
9. Casais R., Dove B., Cavanagh D. & Britton P. Recombinant avian infectious bronchitis virus expressing a heterologous spike gene demonstrates that the spike protein is a determinant of cell tropism. J Virol. 77, 9084-9089(2003).
10. Meeusen, E. N. T., Walker, J., Peters, A., Pastoret, P. P. & Jungersen G. Current status of veterinary vaccines. Clinical Microbiology Reviews. 20, 489-510(2007).
11. Bande, F., Arshad, S. S., Bejo, M. H., Moeini, H. & Omar A R. Progress and challenges toward the development of vaccines against avian infectious bronchitis. Journal of Immunology Research. ID 424860(2015).
12. McKinley, E. T., Hilt, D. A. & Jackwood, M. W. Avian coronavirus infectious bronchitis attenuated live vaccines undergo selection of subpopulations and mutations following vaccination. Vaccine. 26, 1274-1284(2008).
13. Lee, S. W. et al. Attenuated vaccines can recombine to form virulent field viruses.

Science. 337, 188(2012).
14. Nix, W. A., Trorber, D. S., Kingdom, B. F., Keeler, J. C. L. & Gelb, J. J. Emergence of subtype strains of the Arkansas serotype of infectious bronchitis virus in Delmarva broiler chickens. Avian Disease. 44, 568-581(2000).
15. Wakenell, P. S., Sharma, J. M. & Slocombe, R. F. Embryo vaccination of chickens with infectious bronchitis virus: histologic and ultrastructural lesion response and immunologic response to vaccination. Avian Diseases, 39, 752-765(1995).
16. Tarpey, I., Orbell, S. J. & Britton, P. Safety and efficacy of an infectious bronchitis virus used for chicken embryo vaccination. Vaccine, 24, 6830-6838(2006).
17. Collisson, E. W., Pei, J., Dzielawa, J. & Seo, S. H. Cytotoxic T lymphocytes are critical in the control of infectious bronchitis virus in poultry. Developmental & Comparative Immunology. 24, 187-200(2000).
18. Ladman, B. S., Pope, C. R. & Ziegler, A. F. Protection of chickens after live and inactivated virus vaccination against challenge with nephropathogenic infectious bronchitis virus PA/Wolgemuth/98. Avian Diseases. 46, 938-944(2002).
19. Shi, X. M., Zhao, Y. & Gaoetal H. B. Evaluation of recombinant fowl pox virus expressing infectious bronchitis virus S1 gene and chicken interferon-γ gene for immune protection against heterologous strains. Vaccine. 29, 1576-1582(2011).
20. Chen, H. Y., Yang, M. F. & Cui, B. A. Construction and immunogenicity of a recombinant fowl pox vaccine co-expressing S1 glycoprotein of infectious bronchitis virus and chicken IL-18. Vaccine. 28, 8112-8119(2010).
21. Johnson, M. A., Pooley, C., Ignjatovic, J. & Tyack, S. G. A recombinant fowl adenovirus expressing the S1 gene of infectious bronchitis virus protects against challenge with infectious bronchitis virus. Vaccine. 21, 2730-2736(2003).
22. Toro, H. et al. Infectious bronchitis virus S2 expressed from recombinant virus confers broad protection against challenge. Avian Disease. 58, 83-89(2014).
23. Zhao, R. et al. Recombinant Newcastle disease virus expressing the infectious bronchitis virus S1 gene protects chickens against Newcastle disease virus and infectious bronchitis virus challenge. Vaccine. 35, 2435-2442(2017).
24. Samal, S. K. Newcastle disease and related avian paramyxoviruses. In The Biology of Paramyxoviruses. (ed. Samal, S. K.) 69-114 (Norfolk Caister Academic Press, 2011).
25. Kim, S. H. & Samal, S. K. Newcastle disease virus as a vaccine vector for development of human and veterinary vaccines. Viruses. 8, 183(2016).
26. Nayak, B. et al. Immunization of chickens with Newcastle disease virus expressing H5 hemagglutinin protects against highly pathogenic H5N1 avian influenza viruses. PLoS One. 4, 6509 (2009).
27. Schroer, D. et al. Vaccination with Newcastle disease virus vectored vaccine protects chickens against highly pathogenic H7 avian influenza virus. Avian Disease. 53, 190-197(2009).
28. Nagy, A. et al. Recombinant Newcastle disease virus expressing H9 Ha protects chickens against heterologous avian influenza H9N2 virus challenge. Vaccine. 34, 2537-2545(2016).
29. Basavaajappa, M. K. et al. A recombinant Newcastle disease virus (NDV) expressing infectious laryngotracheitis virus (ILTV) surface glycoprotein D protects against highly virulent ILTV and NDV challenges in chickens. Vaccine. 32, 3555-3563(2014).
30. Huang, Z., Krishnamurthy, S., Panda, A. & Samal, S. K. High-level expression of a foreign gene from the most 39-proximal locus of a recombinant Newcastle disease virus. J Gen Virol 82, 1729-1736 (2001).
31. Reed, L. J. & Muench, H. A. A simple method of estimating fifty percent endpoints. Am J Hyg. 27, 493-497 (1983).
32. Calain, P. & Roux, L. The rule of six, a basic feature for efficient replication of Sendai virus defective interfering RNA. J. Virol. 67, 4822-4830(1993).
33. OIE. 2009. Chapter 2.3.14. Newcastle Disease. Version adopted by the World Assembly of Delegates of the OIE in May 2013. In OIE terrestrial manual 2009. www.oie.int/fileadmin/Home/eng/Health_standards/tahm/2.03.14_NEWCASTLE_DIS.pdf.
34. Zhao, W., Zhang, Z., Zsak, L., and Yu, Q. (2015). P and M gene junction is the optimal insertion site in Newcastle disease virus vaccine vector for foreign gene expression. J. Gen. Virol. 96, 40-45. doi: 10.1099/vir.0.068437-0.
35. Yoshida, A. & Samal, S. K. Avian Paramyxovirus type-3 as a vaccine vector: identification of a genome location for high level expression of a foreign gene. Frontiers in Microbiology journal. 8, 693(2017).
36. Eldemery, F., Kellye, S., Toro J. H. & Van Santen, V. L. Protection against infectious bronchitis virus by spike ectodomain subunit vaccine. Vaccine, 35, 5864-5871(2017).

Although the present disclosure has been described with respect to one or more particular embodiments and/or examples, it will be understood that other embodiments and/or examples of the present disclosure may be made without departing from the scope of the present disclosure.

1. A method comprising administering an immunologically effective amount of recombinant Newcastle Disease Virus (rNDV) particles to avian animals to stimulate a protective immune response against infectious bronchitis virus (IBV), wherein the rNDV particles comprises a contiguous segment of IBV S protein that spans an IBV cleavage site between IBV S1 and IBV S2 proteins.

2. The method of claim 1, wherein the contiguous segment of IBV S protein comprises a full length IBV S protein.

3. The method of claim 2, wherein the protective immune response is greater than a reference value obtained from administration of rNDV particles that comprise only an intact S1 or an intact S2 IBV protein, and wherein the protective immune response is determined using a severity score for respiratory clinical signs of IBV infection.

4. The method of claim 2, wherein the protective immune response is serologically distinguishable from an immune response to infection by unmodified IBV.

5. The method of claim 3, wherein RNA encoding the IBV S protein is codon optimized for expression of the IBV S protein in chicken cells.

6. The method of claim 2, wherein the rNDV particles also stimulate a protective immune response against NDV infection.

7. The method of claim 2, wherein the full length IBV S protein is a chimeric protein that further comprises at least a second polypeptide sequence from a pathogen that is not Preliminary Amendment Attorney Docket No.: 070919.00092 NDV or IBV thereby forming at least a multivalent immunogenic agent, and wherein the second polypeptide stimulates a protective immune response to the pathogen that is not NDV or IBV.

8. The method of claim 1, wherein the rNDV particles are administered to an avian animal that is an embryo, a fledgling, or an adult avian animal.

9. The method of claim 7, wherein the avian animal is Gallus gallus.

10. A plurality of isolated rNDV particles of claim 1.

11. The plurality of isolated rNDV particles of claim 10 comprised by a vaccine formulation.

12. The plurality of isolated rNDV particles of claim 10 comprised by an avian embryo.

13. A plurality of rNDV particles of claim 1, wherein the segment of the RNA genome of the rNDV particles encoding the S protein is not mutated such that the amino acid sequence of the S protein encoded by the segment of the RNA sequence is not changed over at least five avian embryo passages.


SEQUENCE LISTING (20 sequences)

SEQ ID NO: 1 - DNA, 54 bp, Newcastle Disease Virus
gtttaaactagctacatttaagattaagaaaaaatacgggtagaagccaccatg

SEQ ID NO: 2 - DNA, 11 bp, Newcastle Disease Virus
taagtttaaac

SEQ ID NO: 3 - protein, 5 aa, Infectious Bronchitis Virus
RRFRR

SEQ ID NO: 4 - protein, 6 aa, Infectious Bronchitis Virus
RRFRRS

SEQ ID NO: 5 - DNA, 20 bp, Artificial Sequence
  Primer
gaccagccgctaacctgaat

SEQ ID NO: 6 - DNA, 20 bp, Artificial Sequence
  Primer
gtcctccgtctgaaaaccgt

SEQ ID NO: 7 - DNA, 15 bp, Newcastle Disease Virus
tagctacatttaaga

SEQ ID NO: 8 - DNA, 11 bp, Newcastle Disease Virus
ttaagaaaaaa

SEQ ID NO: 9 - DNA, 10 bp, Newcastle Disease Virus
acgggtagaa

SEQ ID NO: 10 - protein, 1162 aa, Infectious Bronchitis Virus
MLVTPLLLVTLLCVLCSAALYDSSSYVYYYQSAFRPPNGWHLHGGAYAVVNISSESNNAGSSPGCIVGTIHGGRVVNASSIAMTAPSSGMAWSSSQFCTAHCNFSDTTVFVTHCYKYDGCPITGMLQKNFLRVSAMKNGQLFYNLTVSVAKYPTFKSFQCVNNLTSVYLNGDLVYTSNETTDVTSAGVYFKAGGPITYKVMREVKALAYFVNGTAQDVILCDGSPRGLLACQYNTGNFSDGFYPFINSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPSLCKGVYSGELDLNFECGLLVYVTKSGGSRIQTATEPPVITRHNYNNITLNTCVDYNIYGRTGQGFITNVTDSAVSYNYLADAGLAILDTSGSIDIFVVQGEYGLTYYKVNPCEDVNQQFVVSGGKLVGILTSRNETGSQLLENQFYIKITNGTRRFRRSITENVANCPYVSYGKFCIKPDGSIATIVPKQLEQFVAPLLNVTENVLIPNSFNLTVTDEYIQTRMDKVQINCLQYVCGNSLDCRDLFQQYGPVCDNILSVVNSIGQKEDMELLNFYSSTKPAGFNTPFLSNVSTGEFNISLLLTTPSSPRRRSFIEDLLFTSVESVGLPTDDAYKNCTAGPLGFLKDLACAREYNGLLVLPPIITAEMQTLYTSSLVASMAFGGITAAGAIPFATQLQARINHLGITQSLLLKNQEKIAASFNKAIGRMQEGFRSTSLALQQIQDVVNKQSAILTETMASLNKNFGAISSVIQEIYQQLDAIQANAQVDRLITGRLSSLSVLASAKQAEHIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQANYVSVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQNRPKKSV

SEQ ID NO: 11 - protein, 532 aa, Infectious Bronchitis Virus
MLVTPLLLVTLLCVLCSAALYDSSSYVYYYQSAFRPPNGWHLHGGAYAVVNISSESNNAGSSPGCIVGTIHGGRVVNASSIAMTAPSSGMAWSSSQFCTAHCNFSDTTVFVTHCYKYDGCPITGMLQKNFLRVSAMKNGQLFYNLTVSVAKYPTFKSFQCVNNLTSVYLNGDLVYTSNETTDVTSAGVYFKAGGPITYKVMREVKALAYFVNGTAQDVILCDGSPRGLLACQYNTGNFSDGFYPFINSSLVKQKFIVYRENSVNTTFTLHNFTFHNETGANPNPSGVQNIQTYQTQTAQSGYYNFNFSFLSSFVYKESNFMYGSYHPSCNFRLETINNGLWFNSLSVSIAYGPLQGGCKQSVFSGRATCCYAYSYGGPSLCKGVYSGELDLNFECGLLVYVTKSGGSRIQTATEPPVITRHNYNNITLNTCVDYNIYGRTGQGFITNVTDSAVSYNYLADAGLAILDTSGSIDIFVVQGEYGLTYYKVNPCEDVNQQFVVSGGKLVGILTSRNETGSQLLENQFYIKITNGT

SEQ ID NO: 12 - protein, 6 aa, Infectious Bronchitis Virus
RRFRRS

SEQ ID NO: 13 - protein, 624 aa, Infectious Bronchitis Virus
ITENVANCPYVSYGKFCIKPDGSIATIVPKQLEQFVAPLLNVTENVLIPNSFNLTVTDEYIQTRMDKVQINCLQYVCGNSLDCRDLFQQYGPVCDNILSVVNSIGQKEDMELLNFYSSTKPAGFNTPFLSNVSTGEFNISLLLTTPSSPRRRSFIEDLLFTSVESVGLPTDDAYKNCTAGPLGFLKDLACAREYNGLLVLPPIITAEMQTLYTSSLVASMAFGGITAAGAIPFATQLQARINHLGITQSLLLKNQEKIAASFNKAIGRMQEGFRSTSLALQQIQDVVNKQSAILTETMASLNKNFGAISSVIQEIYQQLDAIQANAQVDRLITGRLSSLSVLASAKQAEHIRVSQQRELATQKINECVKSQSIRYSFCGNGRHVLTIPQNAPNGIVFIHFSYTPDSFVNVTAIVGFCVKPANASQYAIVPANGRGIFIQVNGSYYITARDMYMPRAITAGDIVTLTSCQANYVSVNKTVITTFVDNDDFDFNDELSKWWNDTKHELPDFDKFNYTVPILDIDSEIDRIQGVIQGLNDSLIDLEKLSILKTYIKWPWYVWLAIAFATIIFILILGWVFFMTGCCGCCCGCFGIMPLMSKCGKKSSYYTTFDNDVVTEQNRPKKSV

SEQ ID NO: 14 - DNA, 3548 bp, Artificial Sequence
  codon optimized viral protein coding sequence
gtttaaactagctacatttaagattaagaaaaaatacgggtagaagccaccatgctggtgactccactgctgctggtgactctgctgtgcgtgctgtgttccgccgccctgtacgattcatcatcatacgtgtactattaccagtccgcttttaggccccctaatggatggcacctgcacggcggagcttacgcagtggtgaatatcagctccgagagcaacaatgctggaagctcccctggatgcattgtgggaactattcacggcggcagagtggtgaacgctagctccattgccatgaccgctccaagctccggaatggcctggagctccagccagttctgcacagctcactgcaattttagcgataccacagtgttcgtgacccactgctacaagtacgacggctgcccaatcacaggaatgctgcagaagaactttctgagggtgagcgccatgaaaaacggccagctgttctacaatctgactgtgagcgtggctaagtaccccacctttaaatccttccagtgcgtgaacaatctgaccagcgtgtacctgaacggcgatctggtgtacacaagcaatgagactaccgacgtgacttccgccggagtgtactttaaagctggagggcctatcacatacaaggtgatgagagaagtgaaagccctggcttacttcgtgaacggcactgcacaggatgtgattctgtgcgatggctccccacgcggactgctggcctgccagtacaacaccgggaatttcagcgatggcttttaccccttcatcaactccagcctggtgaagcagaaatttattgtgtacagagagaactccgtgaatacaactttcacactgcacaacttcacttttcacaatgaaaccggcgctaaccccaatcctagcggagtgcagaacatccagacataccagacacagactgcacagagcgggtactacaacttcaatttttccttcctgtccagctttgtgtacaaggagagcaacttcatgtacggcagctaccacccatcctgcaattttcggctggaaacaatcaacaatggactgtggttcaactccctgagcgtgtccattgcatacgggcccctgcagggcggatgcaaacagagcgtgttttccggcagagctacttgctgctacgcatacagctacggcggaccttccctgtgcaagggggtgtacagcggcgagctggatctgaatttcgaatgcggactgctggtgtacgtgaccaaaagcggagggtccaggatccagaccgccacagagccacccgtgatcacacgccacaactacaacaatattactctgaacacctgcgtggactacaatatctacgggaggacaggacaggggttcattacaaacgtgactgatagcgcagtgtcctacaattacctggcagatgctgggctggcaatcctggatactagcggctccatcgacatttttgtggtgcagggagagtacgggctgacctactacaaggtgaacccctgcgaagatgtgaatcagcagttcgtggtgagcggcggaaaactggtgggcatcctgacaagccgcaacgagactggatcccagctgctggaaaaccagttttacatcaagattaccaatggaacaagaaggttcagaagaagcatcaccgagaacgtggctaattgcccctacgtgtcctacggaaagttttgcattaaaccagacgggagcatcgctactattgtgcccaagcagctggagcagtttgtggcacctctgctgaacgtgaccgaaaatgtgctgatccctaacagcttcaatctgactgtgaccgatgaatacatccagacacggatggacaaagtgcagattaactgcctgcagtacgtgtgcggaaatagcctggattgcagagatctgttccagcagtacgggcctgtgtgcgataacatcctgagcgtggtgaattccattggccagaaagaggatatggaactgctgaacttttactccagcaccaaacctgccggctttaacacacctttcctgagcaatgtgtccaccggcgagttcaatatctccctgctgctgaccacaccatccagccccagaaggcgcagctttattgaggatctgctgttcacaagcgtggaatccgtggggctgcccaccgatgacgcatacaagaactgcacagccggccctctgggattcctgaaagacctggcttgcgcacgggagtacaatggcctgctggtgctgcctccaatcattaccgccgaaatgcagactctgtacacctccagcctggtggcaagcatggcctttggcggaatcacagccgctggggccattcccttcgctactcagctgcaggctagaatcaaccacctgggcattactcagtccctgctgctgaagaaccaggagaaaatcgcagccagctttaataaggcaattggacggatgcaggaagggttcagaagcacctccctggccctgcagcagatccaggatgtggtgaacaagcagtccgctattctgacagagactatggcaagcctgaacaaaaattttggcgctatctccagcgtgatccaggaaatttaccagcagctggatgcaatccaggccaacgctcaggtggaccggctgattacaggaagactgtccagcctgagcgtgctggcatccgcaaagcaggctgagcacatcagggtgtcccagcagcgcgagctggctacacagaagatcaacgaatgcgtgaaaagccagtccattcggtacagcttctgcggcaatggaagacacgtgctgactatccctcagaacgcaccaaatggcatcgtgtttattcacttcagctacactccagactcctttgtgaacgtgaccgccatcgtgggattctgcgtgaagccagctaatgcatcccagtacgcaattgtgcccgccaacggcagaggcatctttattcaagtgaatggaagctactacatcactgctagggatatgtacatgcccagagctatcaccgcaggggacattgtgaccctgacaagctgccaggcaaactacgtgtccgtgaataaaaccgtgattactaccttcgtggataacgatgactttgatttcaatgacgagctgtccaagtggtggaacgacacaaaacacgaactgcctgattttgacaagttcaattacactgtgccaatcctggatattgacagcgagatcgaccgcattcagggagtgatccaggggctgaacgatagcctgattgacctggaaaaactgtccatcctgaagacatacattaaatggccttggtacgtgtggctggccatcgcttttgcaaccatcattttcatcctgattctgggatgggtgttctttatgacagggtgctgcggctgctgctgcggatgcttcgggattatgccactgatgagcaagtgcgggaagaaatccagctactatacaacctttgacaatgatgtggtgacagagcagaatcgccctaagaaatccgtgtgagtttaaac

SEQ ID NO: 15 - DNA, 1658 bp, Artificial Sequence
  codon optimized viral protein coding sequence
gtttaaacttaagaaaaaatacgggtagaagccaccatgctggtgactccactgctgctggtgactctgctgtgcgtgctgtgttccgccgccctgtacgattcatcatcatacgtgtactattaccagtccgcttttaggccccctaatggatggcacctgcacggcggagcttacgcagtggtgaatatcagctccgagagcaacaatgctggaagctcccctggatgcattgtgggaactattcacggcggcagagtggtgaacgctagctccattgccatgaccgctccaagctccggaatggcctggagctccagccagttctgcacagctcactgcaattttagcgataccacagtgttcgtgacccactgctacaagtacgacggctgcccaatcacaggaatgctgcagaagaactttctgagggtgagcgccatgaaaaacggccagctgttctacaatctgactgtgagcgtggctaagtaccccacctttaaatccttccagtgcgtgaacaatctgaccagcgtgtacctgaacggcgatctggtgtacacaagcaatgagactaccgacgtgacttccgccggagtgtactttaaagctggagggcctatcacatacaaggtgatgagagaagtgaaagccctggcttacttcgtgaacggcactgcacaggatgtgattctgtgcgatggctccccacgcggactgctggcctgccagtacaacaccgggaatttcagcgatggcttttaccccttcatcaactccagcctggtgaagcagaaatttattgtgtacagagagaactccgtgaatacaactttcacactgcacaacttcacttttcacaatgaaaccggcgctaaccccaatcctagcggagtgcagaacatccagacataccagacacagactgcacagagcgggtactacaacttcaatttttccttcctgtccagctttgtgtacaaggagagcaacttcatgtacggcagctaccacccatcctgcaattttcggctggaaacaatcaacaatggactgtggttcaactccctgagcgtgtccattgcatacgggcccctgcagggcggatgcaaacagagcgtgttttccggcagagctacttgctgctacgcatacagctacggcggaccttccctgtgcaagggggtgtacagcggcgagctggatctgaatttcgaatgcggactgctggtgtacgtgaccaaaagcggagggtccaggatccagaccgccacagagccacccgtgatcacacgccacaactacaacaatattactctgaacacctgcgtggactacaatatctacgggaggacaggacaggggttcattacaaacgtgactgatagcgcagtgtcctacaattacctggcagatgctgggctggcaatcctggatactagcggctccatcgacatttttgtggtgcagggagagtacgggctgacctactacaaggtgaacccctgcgaagatgtgaatcagcagttcgtggtgagcggcggaaaactggtgggcatcctgacaagccgcaacgagactggatcccagctgctggaaaaccagttttacatcaagattaccaatggaacaagaaggttcagaagatgagtttaaac

SEQ ID NO: 16 - DNA, 1928 bp, Artificial Sequence
  codon optimized viral protein coding sequence
gtttaaactagctacatttaagattaagaaaaaatacgggtagaagccaccatgctggtgactccactgctgctggtgactctgctgtgcgtgctgtgttccgccgccctgtacgattcatcatcatacgtgtactattaccagtccgcttttaggccccctaatggatggcacctgcacggcggagcttacgcagtggtgaatatcagctccgagagcaacaatgctggaagctcccctggatgcattgtgggaactattcacggcggcagagtggtgaacgctagctccattgccatgaccgctccaagctccggaatggcctggagctccagccagttctgcacagctcactgcaattttagcgataccacagtgttcgtgacccactgctacaagtacgacggctgcccaatcacaggaatgctgcagaagaactttctgagggtgagcgccatgaaaaacggccagctgttctacaatctgactgtgagcgtggctaagtaccccacctttaaatccttccagtgcgtgaacaatctgaccagcgtgtacctgaacggcgatctggtgtacacaagcaatgagactaccgacgtgacttccgccggagtgtactttaaagctggagggcctatcacatacaaggtgatgagagaagtgaaagccctggcttacttcgtgaacggcactgcacaggatgtgattctgtgcgatggctccccacgcggactgctggcctgccagtacaacaccgggaatttcagcgatggcttttaccccttcatcaactccagcctggtgaagcagaaatttattgtgtacagagagaactccgtgaatacaactttcacactgcacaacttcacttttcacaatgaaaccggcgctaaccccaatcctagcggagtgcagaacatccagacataccagacacagactgcacagagcgggtactacaacttcaatttttccttcctgtccagctttgtgtacaaggagagcaacttcatgtacggcagctaccacccatcctgcaattttcggctggaaacaatcaacaatggactgtggttcaactccctgagcgtgtccattgcatacgggcccctgcagggcggatgcaaacagagcgtgttttccggcagagctacttgctgctacgcatacagctacggcggaccttccctgtgcaagggggtgtacagcggcgagctggatctgaatttcgaatgcggactgctggtgtacgtgaccaaaagcggagggtccaggatccagaccgccacagagccacccgtgatcacacgccacaactacaacaatattactctgaacacctgcgtggactacaatatctacgggaggacaggacaggggttcattacaaacgtgactgatagcgcagtgtcctacaattacctggcagatgctgggctggcaatcctggatactagcggctccatcgacatttttgtggtgcagggagagtacgggctgacctactacaaggtgaacccctgcgaagatgtgaatcagcagttcgtggtgagcggcggaaaactggtgggcatcctgacaagccgcaacgagactggatcccagctgctggaaaaccagttttacatcaagattaccaatggaacaagaaggttcagaagaattgacctggaaaaactgtccatcctgaagacatacattaaatggccttggtacgtgtggctggccatcgcttttgcaaccatcattttcatcctgattctgggatgggtgttctttatgacagggtgctgcggctgctgctgcggatgcttcgggattatgccactgatgagcaagtgcgggaagaaatccagctactatacaacctttgacaatgatgtggtgacagagcagaatcgccctaagaaatccgtgtgagtttaaac

SEQ ID NO: 17 - DNA, 1844 bp, Artificial Sequence
  codon optimized viral protein coding sequence
gtttaaactagctacatttaagattaagaaaaaatacgggtagaagccaccatgctggtgactccactgctgctggtgactctgctgtgcgtgctgtgttccgccgccctgtacgattcatcatcatacgtgtactattaccagtccgcttttaggccccctaatggatggcacctgcacggcggagcttacgcagtggtgaatatcagctccgagagcaacaatgctggaagctcccctggatgcattgtgggaactattcacggcggcagagtggtgaacgctagctccattgccatgaccgctccaagctccggaatggcctggagctccagccagttctgcacagctcactgcaattttagcgataccacagtgttcgtgacccactgctacaagtacgacggctgcccaatcacaggaatgctgcagaagaactttctgagggtgagcgccatgaaaaacggccagctgttctacaatctgactgtgagcgtggctaagtaccccacctttaaatccttccagtgcgtgaacaatctgaccagcgtgtacctgaacggcgatctggtgtacacaagcaatgagactaccgacgtgacttccgccggagtgtactttaaagctggagggcctatcacatacaaggtgatgagagaagtgaaagccctggcttacttcgtgaacggcactgcacaggatgtgattctgtgcgatggctccccacgcggactgctggcctgccagtacaacaccgggaatttcagcgatggcttttaccccttcatcaactccagcctggtgaagcagaaatttattgtgtacagagagaactccgtgaatacaactttcacactgcacaacttcacttttcacaatgaaaccggcgctaaccccaatcctagcggagtgcagaacatccagacataccagacacagactgcacagagcgggtactacaacttcaatttttccttcctgtccagctttgtgtacaaggagagcaacttcatgtacggcagctaccacccatcctgcaattttcggctggaaacaatcaacaatggactgtggttcaactccctgagcgtgtccattgcatacgggcccctgcagggcggatgcaaacagagcgtgttttccggcagagctacttgctgctacgcatacagctacggcggaccttccctgtgcaagggggtgtacagcggcgagctggatctgaatttcgaatgcggactgctggtgtacgtgaccaaaagcggagggtccaggatccagaccgccacagagccacccgtgatcacacgccacaactacaacaatattactctgaacacctgcgtggactacaatatctacgggaggacaggacaggggttcattacaaacgtgactgatagcgcagtgtcctacaattacctggcagatgctgggctggcaatcctggatactagcggctccatcgacatttttgtggtgcagggagagtacgggctgacctactacaaggtgaacccctgcgaagatgtgaatcagcagttcgtggtgagcggcggaaaactggtgggcatcctgacaagccgcaacgagactggatcccagctgctggaaaaccagttttacatcaagattaccaatggaacaagaaggttcagaagaagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgagtttaaac

SEQ ID NO: 18 - DNA, 1826 bp, Artificial Sequence
  codon optimized viral protein coding sequence
gtttaaactagctacatttaagattaagaaaaaatacgggtagaagccaccatgctggtgactccactgctgctggtgactctgctgtgcgtgctgtgttccgccgccctgtacgattcatcatcatacgtgtactattaccagtccgcttttaggccccctaatggatggcacctgcacggcggagcttacgcagtggtgaatatcagctccgagagcaacaatgctggaagctcccctggatgcattgtgggaactattcacggcggcagagtggtgaacgctagctccattgccatgaccgctccaagctccggaatggcctggagctccagccagttctgcacagctcactgcaattttagcgataccacagtgttcgtgacccactgctacaagtacgacggctgcccaatcacaggaatgctgcagaagaactttctgagggtgagcgccatgaaaaacggccagctgttctacaatctgactgtgagcgtggctaagtaccccacctttaaatccttccagtgcgtgaacaatctgaccagcgtgtacctgaacggcgatctggtgtacacaagcaatgagactaccgacgtgacttccgccggagtgtactttaaagctggagggcctatcacatacaaggtgatgagagaagtgaaagccctggcttacttcgtgaacggcactgcacaggatgtgattctgtgcgatggctccccacgcggactgctggcctgccagtacaacaccgggaatttcagcgatggcttttaccccttcatcaactccagcctggtgaagcagaaatttattgtgtacagagagaactccgtgaatacaactttcacactgcacaacttcacttttcacaatgaaaccggcgctaaccccaatcctagcggagtgcagaacatccagacataccagacacagactgcacagagcgggtactacaacttcaatttttccttcctgtccagctttgtgtacaaggagagcaacttcatgtacggcagctaccacccatcctgcaattttcggctggaaacaatcaacaatggactgtggttcaactccctgagcgtgtccattgcatacgggcccctgcagggcggatgcaaacagagcgtgttttccggcagagctacttgctgctacgcatacagctacggcggaccttccctgtgcaagggggtgtacagcggcgagctggatctgaatttcgaatgcggactgctggtgtacgtgaccaaaagcggagggtccaggatccagaccgccacagagccacccgtgatcacacgccacaactacaacaatattactctgaacacctgcgtggactacaatatctacgggaggacaggacaggggttcattacaaacgtgactgatagcgcagtgtcctacaattacctggcagatgctgggctggcaatcctggatactagcggctccatcgacatttttgtggtgcagggagagtacgggctgacctactacaaggtgaacccctgcgaagatgtgaatcagcagttcgtggtgagcggcggaaaactggtgggcatcctgacaagccgcaacgagactggatcccagctgctggaaaaccagttttacatcaagattaccaatggaagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgagtttaaac

SEQ ID NO: 19 - DNA, 2006 bp, Artificial Sequence
  codon optimized viral protein coding sequence
gtttaaactagctacatttaagattaagaaaaaatacgggtagaagccaccatgctggtgactccactgctgctggtgactctgctgtgcgtgctgtgttccgccgccctgtacgattcaagcatcaccgagaacgtggctaattgcccctacgtgtcctacggaaagttttgcattaaaccagacgggagcatcgctactattgtgcccaagcagctggagcagtttgtggcacctctgctgaacgtgaccgaaaatgtgctgatccctaacagcttcaatctgactgtgaccgatgaatacatccagacacggatggacaaagtgcagattaactgcctgcagtacgtgtgcggaaatagcctggattgcagagatctgttccagcagtacgggcctgtgtgcgataacatcctgagcgtggtgaattccattggccagaaagaggatatggaactgctgaacttttactccagcaccaaacctgccggctttaacacacctttcctgagcaatgtgtccaccggcgagttcaatatctccctgctgctgaccacaccatccagccccagaaggcgcagctttattgaggatctgctgttcacaagcgtggaatccgtggggctgcccaccgatgacgcatacaagaactgcacagccggccctctgggattcctgaaagacctggcttgcgcacgggagtacaatggcctgctggtgctgcctccaatcattaccgccgaaatgcagactctgtacacctccagcctggtggcaagcatggcctttggcggaatcacagccgctggggccattcccttcgctactcagctgcaggctagaatcaaccacctgggcattactcagtccctgctgctgaagaaccaggagaaaatcgcagccagctttaataaggcaattggacggatgcaggaagggttcagaagcacctccctggccctgcagcagatccaggatgtggtgaacaagcagtccgctattctgacagagactatggcaagcctgaacaaaaattttggcgctatctccagcgtgatccaggaaatttaccagcagctggatgcaatccaggccaacgctcaggtggaccggctgattacaggaagactgtccagcctgagcgtgctggcatccgcaaagcaggctgagcacatcagggtgtcccagcagcgcgagctggctacacagaagatcaacgaatgcgtgaaaagccagtccattcggtacagcttctgcggcaatggaagacacgtgctgactatccctcagaacgcaccaaatggcatcgtgtttattcacttcagctacactccagactcctttgtgaacgtgaccgccatcgtgggattctgcgtgaagccagctaatgcatcccagtacgcaattgtgcccgccaacggcagaggcatctttattcaagtgaatggaagctactacatcactgctagggatatgtacatgcccagagctatcaccgcaggggacattgtgaccctgacaagctgccaggcaaactacgtgtccgtgaataaaaccgtgattactaccttcgtggataacgatgactttgatttcaatgacgagctgtccaagtggtggaacgacacaaaacacgaactgcctgattttgacaagttcaattacactgtgccaatcctggatattgacagcgagatcgaccgcattcagggagtgatccaggggctgaacgatagcctgattgacctggaaaaactgtccatcctgaagacatacattaaatggccttggtacgtgtggctggccatcgcttttgcaaccatcattttcatcctgattctgggatgggtgttctttatgacagggtgctgcggctgctgctgcggatgcttcgggattatgccactgatgagcaagtgcgggaagaaatccagctactatacaacctttgacaatgatgtggtgacagagcagaatcgccctaagaaatccgtgtgagtttaaac

SEQ ID NO: 20 - protein, 553 aa, Newcastle Disease Virus
MGSRPSTKNPAPMMLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGVGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM